(19) United States
(12) Patent Application Publication
(10) Pub. No.: US 2026/0347854 A1
(43) Pub. Date: Nov. 5, 2026

(54) ALLELE SPECIFIC SIRNA THERAPY FOR DYNAMIN 2-RELATED DISEASES

(71) Applicants: INSTITUT NATIONAL DE LA SANTÉ ET DE LA RECHERCHE MÉDICALE, PARIS (FR); SORBONNE UNIVERSITÉ, PARIS (FR); ASSOCIATION INSTITUT DE MYOLOGIE, PARIS (FR)

(72) Inventors: Marc BITOUN, PARIS CEDEX 13 (FR); Delphine TROCHET, PARIS CEDEX 13 (FR)

(21) Appl. No.: 19/102,548

(22) PCT Filed: Aug. 10, 2023
(86) PCT No.: PCT/EP2023/072157

§ 371 Date: Feb. 10, 2025

(30) Foreign Application Priority Data
Aug. 11, 2022 (EP) 22306213.4

(51) Int. Cl. C12N 15/113; A61K 31/7105; A61P 21/00; C12N 15/85

(52) U.S. Cl. CPC C12N 15/1137; A61K 31/7105; A61P 21/00; C12N 15/85; C12N 2310/14; C12N 2320/34; C12Y 306/05005

(57) ABSTRACT

The present invention relates to the treatment of myopathy. The inventors report the identification of effective AS-siRNA against the two nucleotide versions of the two non-pathogenic DNM2 SNPs which may be used to silence any mutation carried by the same mRNA. In addition, the first AS-siRNAs targeting a DNM2 mutation associated with severe neonatal phenotype, i.e. the p.S619L mutation, have been developed and in a second time, they developed other AS-siRNA targeting the mutation p. S619L and the mutation p.R465W. They also report the functional benefits of this new set of siRNAs on several defects identified in patient-derived cell lines. The development of these new AS-siRNA, in addition to the previous ones against the p.R465W mutation, provides a panoply of allele-specific molecules able to target the large majority of AD-CNM patients. Interestingly, siRNAs against the DNM2 SNPs represent versatile molecules with larger potential applications to silence DNM2 mutations in CMT and HSP and to reduce DNM2 expression in a controlled manner in diseases associated with deleterious overexpression. Thus, the present invention relates to an allele specific siRNA (AS-siRNA) able to silence the expression of only one allele of a heterozygous DNM2 gene wherein the targeted allele comprises a non-pathological polymorphism selected from the group consisting of rs2229920 (C or T) or rs12461992 (A or T) and/or a disease-causing mutation selected from the group consisting of c.1393C>T or c.1856C>T.

### FIELD OF THE INVENTION

The present invention relates to an allele specific siRNA (AS-siRNA) able to silence the expression of only one allele of a heterozygous DNM2 gene wherein the targeted allele comprises a non-pathological polymorphism selected from the group consisting of rs2229920 (C or T) or rs12461992 (A or T) and/or a disease-causing mutation selected from the group consisting of c.1393C>T or c.1856C>T.

### BACKGROUND OF THE INVENTION

Autosomal dominant centronuclear myopathy (AD-CNM, MIM #160150) is a rare congenital myopathy associated with a wide clinical spectrum from severe-neonatal to mild-adult forms (1). The classical late-childhood or adult-onset form exhibits delayed motor milestones and diffuse skeletal muscle weakness mainly involving facial and limb muscles whereas paediatric patients affected by the severe neonatal form usually have generalized weakness, hypotonia, and facial weakness (2,3). AD-CNM results from mutations in the DNM2 gene which encodes dynamin 2 (DNM2) (4). In addition, DNM2 mutations also cause rare forms of Charcot-Marie-Tooth disease (CMT) (5) and hereditary spastic paraplegia (HSP) (6) and a deleterious DNM2 overexpression was reported in several cancers (7-12) and the X-linked recessive CNM (13), highlighting a large DNM2 involvement in human diseases. DNM2 belongs to the superfamily of large GTPases (14) acting as a mechanochemical scaffolding molecule that oligomerizes and deforms biological membranes leading to the formation and release of vesicles from the plasma membrane and intracellular membrane compartments. Furthermore, several studies have highlighted the role of DNM2 as a regulator of both actin and microtubule cytoskeletons (15,16). More than 30 DNM2 mutations have been reported in AD-CNM patients (17) and, when tested, the mutant protein is normally expressed (4,18). Mutations are thought to be responsible for a gain of function and/or a dominant-negative effect through an increased GTPase activity and formation of abnormal stable DNM2 oligomers (19,20). In addition, absence of haploinsufficiency in AD-CNM is supported by data from patients and absence of phenotype developed by heterozygous knockout mice expressing 50% Dnm2 (13,21).

The inventors recently developed a therapeutic approach for the DNM2-related AD-CNM by allele-specific RNA interference (AS-RNAi) devoted to specifically suppressing the expression of the mutated protein from the mutated allele without reducing expression from the wild-type allele (22). AS-RNAi was demonstrated as a powerful strategy in cells from patients and animal models of numerous dominant inherited diseases (23) and led to two clinical trials targeting a Keratin 6a mutation causing Pachyonychia congenita (24) and one mutation of the KRAS gene involved in pancreatic cancer (25). By applying this strategy, they reported functional rescue in the Knock-In-Dnm2R465W/+ mouse model of AD-CNM and patient-derived fibroblasts, both expressing the most frequent mutation encountered in patients (p.R465W found in around 30% of patients) (22). Extending this strategy to the entire AD-CNM patient population requires either to develop personalized medicine through specific siRNA for each reported mutation or to develop an alternative approach allowing to target the distinct dominant DNM2 mutations using a limited number of allele-specific siRNA. In particular cases of triplet repeat diseases such as Huntington's disease or spinocerebellar ataxia in which the sequence of the genetic mutations makes allele-specific silencing challenging, AS-siRNA have been developed against disease-associated single nucleotide polymorphisms (SNP) (23, 26-28). They took advantage of the presence in the DNM2 sequence of two non-pathogenic SNPs frequently heterozygous in the general population to develop a similar strategy for the AD-CNM.

### SUMMARY OF THE INVENTION

Here, the inventors report the identification of effective AS-siRNA against the two nucleotide versions of the two non-pathogenic DNM2 SNPs which may be used to silence any mutation carried by the same mRNA. In addition, the first AS-siRNAs targeting a DNM2 mutation associated with severe neonatal phenotype, i.e. the p.S619L mutation, have been developed and in a second time, they developed other AS-siRNA targeting the mutation p.S619L and the mutation p.R465W. They also report the functional benefits of this new set of siRNAs on several defects identified in patient-derived cell lines. The development of these new AS-siRNA, in addition to the previous ones against the p.R465W mutation, provides a panoply of allele-specific molecules able to target the large majority of AD-CNM patients. Interestingly, siRNAs against the DNM2 SNPs represent versatile molecules with larger potential applications to silence DNM2 mutations in CMT and HSP and to reduce DNM2 expression in a controlled manner in diseases associated with deleterious overexpression.

Thus, the present invention relates to an allele specific siRNA (AS-siRNA) able to silence the expression of only one allele of a heterozygous DNM2 gene wherein the targeted allele comprises a non-pathological polymorphism selected from the group consisting of rs2229920 (C or T) or rs12461992 (A or T) and/or a disease-causing mutation selected from the group consisting of c.1393C>T or c.1856C>T. Particularly, the invention is defined by its claims.

### DETAILED DESCRIPTION OF THE INVENTION

The inventors have investigated a therapeutic approach based on the specific suppression of the expression of only one allele of DNM2, preserving the other DNM2 allele. On one hand, this strategy aims to reduce in a controlled way the DNM2 expression level, in the case of diseases related to overexpression of DNM2. On the other hand, this strategy would be useful in autosomal dominant inherited diseases due to heterozygous mutation in the DNM2 gene, by specifically inhibiting the expression of a mutant allele without reducing the level of the wild type DNM2 allele which is required for a normal cellular function. With this objective, the present inventors discovered very efficient allele specific siRNAs able to inhibit, in a controlled way, only one allele of a heterozygous DNM2 gene in a cell. This strategy was patented in the patent application WO2018100010. However, the inventors didn't design and test all siRNA possible against the two most frequent non-pathogenic DNM2 SNPs rs2229920 (C or T) or rs12461992 (A or T) and the two most disease-causing mutations c.1393C>T or c.1856C>T. In the present application, the inventors design these siRNAs and tested them. They showed that some are very effective both in-vitro and in-vivo.

In a first aspect, the invention relates to an allele specific siRNA (AS-siRNA) able to silence the expression of only one allele of a heterozygous DNM2 gene wherein the targeted allele comprises a non-pathological polymorphism selected from the group consisting of rs2229920 (C or T) or rs12461992 (A or T) and/or a disease-causing mutation selected from the group consisting of c.1393C>T or c.1856C>T.

The Dynamin 2 is encoded by the DNM2 gene (Gene ID 1785). More precisely, the DNM2 gene is located within the short arm of chromosome 19 at position 13.2 (19p13.2). The dynamin 2 gene or gene products are also known by other names, including but not limited to CMT2M, CMTDI1, CMTDIB, DI-CMTB, DYN2, DYN2_HUMAN, dynamin II, DYNII. DNM2 has an important role in endocytosis and in the cell's structural framework (cytoskeleton). The protein interacts with multiple parts of the cytoskeleton, including microtubules and actin, which organize into filaments to provide structure. These parts of the cytoskeleton are involved in movement of molecules within the cells, cell shape, cell mobility, and attachment of cells to one another or to extracellular matrix. An alteration in the DNM2 gene may thus disrupt endocytosis and interfere with the arrangement or dynamics of cytoskeletons leading to abnormal cellular function. As previously described, several dominant genetic diseases are caused by heterozygous mutations of the DNM2 gene such as autosomal dominant centronuclear myopathy, Charcot-Marie-Tooth disease and Hereditary Spastic Paraplegia. Overexpression of DNM2 is also pathological and involved in some pathophysiological mechanisms of other diseases such as X-linked myotubular myopathy or cancers, for example prostate cancer and pancreatic cancer.

RNA interference is a biological process in which RNA molecules inhibit gene expression, typically by causing the destruction of specific mRNA molecules. An interfering RNA is therefore an RNA which is capable of down-regulating the expression of the targeted protein. For example, it encompasses small interfering RNA (siRNA), double-stranded RNA (dsRNA), single-stranded RNA (ssRNA), and short hairpin RNA (shRNA) molecules. RNA interference designates a phenomenon by which dsRNA specifically suppresses expression of a target gene at post-transcriptional level. In normal conditions, RNA interference is initiated by double-stranded RNA molecules (dsRNA) of several thousands of base pair length. In vivo, a dsRNA introduced into a cell is cleaved by an enzyme called DICER into a mixture of short dsRNA molecules called siRNA. In mammalian cells, the siRNAs produced by Dicer are about 21 base-pairs (bp) in length. Then the siRNA join an RNase complex, RISC (RNA-induced silencing complex), which acts on the cognate mRNA and degrades it. RNA interference is also a valuable research tool, as double strand siRNA of 19 to 23 bp may be used to selectively and robustly induce suppression of specific genes of interest. The major interest of this approach is the specificity, as an siRNA is able to discriminate two sequences even when differing by only a single nucleotide.

The present inventors used said specificity of siRNA to specifically inhibit one allele of a heterozygous DNM2 gene. Consequently, in that particular case, the siRNA is called an “allele specific siRNA” (AS-siRNA).

By AS-siRNA is meant any siRNA able to specifically silence only one allele of a targeted gene, an allele being one of several alternative forms of a gene occupying a given locus on a chromosome. “Gene silencing” refers to the suppression or reduction of gene expression. Gene silencing may be mediated through processes that affect transcription and/or through processes that affect post-transcriptional mechanisms. In some embodiments, gene silencing occurs when siRNA initiates the degradation of the mRNA of the gene in a sequence-specific manner via RNA interference. Thus, a gene includes coding sequences and/or the regulatory sequences required for expression. For example, “gene” refers to a nucleic acid fragment that expresses mRNA, functional RNA, or specific protein, including regulatory sequences. “Genes” also include nonexpressed DNA segments that may, for example, form recognition sequences for other proteins.

In the context of the invention, the gene is the DNM2 gene, coding for Dynamin 2 protein. Thus, an AS-siRNA of the invention specifically silences one allele of the DNM2 gene, which is a variant form of the DNM2 gene.

In the context of the present invention, the DNM2 gene is a heterozygous DNM2 gene. A heterozygous DNM2 gene is a DNM2 gene present in a heterozygous state in a cell. By “heterozygous” is meant that a given chromosomal locus has two different alleles. Diploid organisms such as humans contain two copies of each chromosome (one maternal and one paternal chromosome), that are called homologous chromosomes. Therefore, each homologous chromosome carries one allele of a given gene. A diploid organism is heterozygous when said two alleles of a given gene are different in respect to a given variation or polymorphism.

In the context of the invention, a cell or an organism is heterozygous in respect to the DNM2 gene wherein the DNM2 gene is present in a heterozygous state, that is to say wherein the two alleles of DNM2 gene are different in respect to a given variation or polymorphism.

In one embodiment of the invention, heterozygous refers to a genotype in which one allele has a wild-type DNM2 sequence and the other allele has a sequence encoding a DNM2 variant. In particular, the sequence encoding a DNM2 variant comprises a mutation that is not present in the wild-type sequence.

In a particular embodiment, the DNM2 gene is heterozygous for the presence of a non-pathological polymorphism. In this second embodiment, the AS-siRNA of the invention targets only one of the allele of the DNM2 gene, comprising or not said non-pathological polymorphism.

In a particular embodiment, the DNM2 gene is heterozygous for the presence of a disease-causing mutation. In this embodiment, the AS-siRNA of the invention targets the allele of the DNM2 gene comprising said disease-causing mutation.

### As-siRNA Targeting the Allele of DNM2 Gene Comprising a Non-Pathological Polymorphism

In a particular aspect, the invention relates to an AS-siRNA able to silence the expression of only one allele of a heterozygous DNM2 gene in a cell, wherein the DNM2 gene is heterozygous for the presence of a non-pathological polymorphism.

In a particular embodiment, the DNM2 gene comprises a common heterozygous non-pathological polymorphism. “Common heterozygous non pathological polymorphism” refers to a polymorphism with high heterozygous frequency that is to say that is frequent in the population at heterozygous state. By “frequent” is meant a polymorphism which is found at heterozygous state in at least 20%, 30%, 40% of general population, particularly at least 40%.

By “non-pathological polymorphism” is meant a variation in the nucleic acid sequence of a gene that is not associated with a disease. As such, according to the present invention, a non-pathological polymorphism corresponds to a sequence variation in a gene that, when considered independently of other sequence modifications, is not by itself associated to a pathology. For the sake of clarity, if a non-pathological polymorphism is heterozygous in a cell, it means that both polymorphisms are considered non-pathological, if considered independently of other sequence variations that might occur on the same gene. Non-pathological polymorphisms may include variations in coding and non-coding regions. Furthermore, non-pathological polymorphisms include nucleotide substitutions, deletions, and/or additions, including those that result in missense and nonsense mutations which do not lead to a pathology. Particularly, the non-pathological polymorphism is a single nucleotide substitution.

By targeting heterozygous common polymorphisms, the AS-siRNA of the invention can be used to reduce the expression of DNM2 protein, especially when overexpression of DNM2 in absence of mutation is associated with a pathological condition. For example, overexpression of DNM2 protein, in absence of mutation is correlated to X-linked myotubular myopathy or cancer such as prostate cancer and pancreatic cancer. Thus, the invention relates to an AS-siRNA wherein the AS-siRNA targets a DNM2 allele comprising a non-pathological polymorphism.

In another embodiment, the DNM2 allele comprising a non-pathological polymorphism is on the same allele as a heterozygous disease-causing mutation. By targeting heterozygous common polymorphisms rather than each specific disease-causing mutation, a single AS-siRNA can be used to inhibit expression of more than one disease-causing mutation in more than one patient. Accordingly, in one embodiment, the targeted version of the heterozygous non-pathological polymorphism is present on the same allele as said disease-causing mutation and is absent on the wild type allele which harbors the other version of the polymorphism. In other words, targeting a heterozygous non-pathological polymorphism allows differentiating mutant and wild-type DNM2 alleles. Said disease-causing mutation can be any heterozygous mutation within the DNM2 gene responsible for or associated to a disease. For example, the disease-causing mutation within the DNM2 gene is responsible for or correlated to a disease selected from Autosomal Dominant Centronuclear Myopathy (AD-CNM), T-cell acute lymphoblastic leukemia, Charcot-Marie-Tooth disease (CMT) or Hereditary Spastic Paraplegia (HSP). Particularly, the disease-causing mutation within the DNM2 gene is responsible for or correlated to Autosomal Dominant Centronuclear Myopathy (AD-CNM). In a particular embodiment, the DNM2 gene is heterozygous for the presence of a missense mutation selected in the group consisting of: c.1393C>T; c.1105C>T; c.1106G>A; c.1565G>A; c.1856C>T or c.1948G>A, respectively responsible for the following substitution in the DNM2 protein sequence: p.R465W, p.R369W, p.R369Q, p.R522H, p.S619L, and p.E650K. In another particular embodiment, the DNM2 gene is heterozygous for the presence of the c.1393C>T mutation, responsible for the p.R465W substitution in the DNM2 protein sequence.

Thus, in a particular aspect, the invention relates to an AS-siRNA able to silence the expression of only one allele of a heterozygous DNM2 gene in a cell, wherein the targeted allele comprises a non-pathological polymorphism selected from the group consisting of rs2229920 (C or T) or rs12461992 (A or T).

The inventors have indeed designed many siRNA able to target the most frequent non-pathological polymorphisms (rs2229920 (C or T) called SNP1 or rs12461992 (A or T) called SNP2) and tested in in-vitro and in-vivo experiments. These sequences are listed in the tables 1 to 4.

Thus, the invention also relates to an allele specific siRNA (AS-siRNA) able to silence the expression of only one allele of a heterozygous DNM2 gene wherein the AS-siRNA comprises a sense strand selected in the group consisting of SEQ ID NO: 1, 3, 5, 7, 9, 11, 13, 15, 17, 19, 21, 23, 25, 27, 29, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149 and 151.

Particularly, the invention relates to an allele specific siRNA (AS-siRNA) able to silence the expression of only one allele of a heterozygous DNM2 gene wherein the AS-siRNA comprises a sense strand selected in the group consisting of SEQ ID NO: 15, 17, 19, 33, 51, 53, 55, 57, 61, 67, 69, 71, 93, 95, 97, 135, 137, 141, 143, 145 and 147.

TABLE 1

| | | Sequence |
|---|---|---|
| Name | siRNA sequence | number |
| Si1-SNP1-T-sens | 5′-UGACCAGGCACAGCGGCGG-3′ | SEQ ID NO: 1 |
| Si1-SNP1-T-antisense | 3′-ACUGGUCCGUGUCGCCGCC-5′ | SEQ ID NO: 2 |
| Si2-SNP1-T-sense | CUGACCAGGCACAGCGGCG | SEQ ID NO: 3 |
| Si2-SNP1-T-antisense | GACUGGUCCGUGUCGCCGC | SEQ ID NO: 4 |
| Si3-SNP1-T-sense | GCUGACCAGGCACAGCGGC | SEQ ID NO: 5 |
| Si3-SNP1-T-antisense | CGACUGGUCCGUGUCGCCG | SEQ ID NO: 6 |
| Si4-SNP1-T-sense | GGCUGACCAGGCACAGCGG | SEQ ID NO: 7 |
| Si4-SNP1-T-antisense | CCGACUGGUCCGUGUCGCC | SEQ ID NO: 8 |
| Si5-SNP1-T-sense | CGGCUGACCAGGCACAGCG | SEQ ID NO: 9 |
| Si5-SNP1-T-antisens | GCCGACUGGUCCGUGUCGC | SEQ ID NO: 10 |
| Si6-SNP1-T-sense | UCGGCUGACCAGGCACAGC | SEQ ID NO: 11 |
| Si6-SNP1-T-antisense | AGCCGACUGGUCCGUGUCG | SEQ ID NO: 12 |
| Si7-SNP1-T-sense | GUCGGCUGACCAGGCACAG | SEQ ID NO: 13 |
| Si7-SNP1-T-antisense | CAGCCGACUGGUCCGUGUC | SEQ ID NO: 14 |
| Si8-SNP1-T-sense | AGUCGGCUGACCAGGCACA | SEQ ID NO: 15 |
| Si8-SNP1-T-antisense | UCAGCCGACUGGUCCGUGU | SEQ ID NO: 16 |
| Si9-SNP1-T-sense | GAGUCGGCUGACCAGGCAC | SEQ ID NO: 17 |
| Si9-SNP1-T-antisense | CUCAGCCGACUGGUCCGUG | SEQ ID NO: 18 |
| Si10-SNP1-T-sense | GGAGUCGGCUGACCAGGCA | SEQ ID NO: 19 |
| Si10-SNP1-T-antisense | CCUCAGCCGACUGGUCCGU | SEQ ID NO: 20 |
| Si11-SNP1-T-sense | AGGAGUCGGCUGACCAGGC | SEQ ID NO: 21 |
| Si11-SNP1-T-antisense | UCCUCAGCCGACUGGUCCG | SEQ ID NO: 22 |
| Si12-SNP1-T-sense | GAGGAGUCGGCUGACCAGG | SEQ ID NO: 23 |
| Si12-SNP1-T-antisense | CUCCUCAGCCGACUGGUCC | SEQ ID NO: 24 |
| Si13-SNP1-T-sense | GGAGGAGUCGGCUGACCAG | SEQ ID NO: 25 |
| Si13-SNP1-T-antisense | CCUCCUCAGCCGACUGGUC | SEQ ID NO: 26 |
| Si14-SNP1-T-sense | UGGAGGAGUCGGCUGACCA | SEQ ID NO: 27 |
| Si14-SNP1-T-antisense | ACCUCCUCAGCCGACUGGU | SEQ ID NO: 28 |
| Si15-SNP1-T-sense | AUGGAGGAGUCGGCUGACC | SEQ ID NO: 29 |
| Si15-SNP1-T-antisense | UACCUCCUCAGCCGACUGG | SEQ ID NO: 30 |
| Si16-SNP1-T-sense | CAUGGAGGAGUCGGCUGAC | SEQ ID NO: 31 |
| Si16-SNP1-T-antisense | GUACCUCCUCAGCCGACUG | SEQ ID NO: 32 |
| Si17-SNP1-T-sense | UCAUGGAGGAGUCGGCUGA | SEQ ID NO: 33 |
| Si17-SNP1-T-antisense | AGUACCUCCUCAGCCGACU | SEQ ID NO: 34 |
| Si18-SNP1-T-sense | CUCAUGGAGGAGUCGGCUG | SEQ ID NO: 35 |
| Si18-SNP1-T-antisense | GAGUACCUCCUCAGCCGAC | SEQ ID NO: 36 |
| Si19-SNP1-T-sense | CCUCAUGGAGGAGUCGGCU | SEQ ID NO: 37 |
| Si19-SNP1-T-antisense | GGAGUACCUCCUCAGCCGA | SEQ ID NO: 38 |

siRNA against the SNP1 humain (rs2229920) with a polymorphism T. The sequence at the top corresponds to the sense strand (or the sense sequence) (5′-3′) and is call “passenger sequence” and the at the bottom corresponds to the antisense strand (or the antisense sequence) (3′-5′) and is call “guide sequence” (see the Si1-SNP1-T for example).

TABLE 2

| | | Sequence |
|---|---|---|
| Name | siRNA sequence | number |
| Si1-SNP1-C-sense | 5′-CGACCAGGCACAGCGGCGG-3′ | SEQ ID NO: 39 |
| Si1-SNP2-C-antisense | 3′-GCUGGUCCGUGUCGCCGCC-5′ | SEQ ID NO: 40 |
| Si2-SNP1-C-sense | CCGACCAGGCACAGCGGCG | SEQ ID NO: 41 |
| Si2-SNP1-C-antisense | GGCUGGUCCGUGUCGCCGC | SEQ ID NO: 42 |
| Si3-SNP1-C-sense | GCCGACCAGGCACAGCGGC | SEQ ID NO: 43 |
| Si3-SNP1-C-antisense | CGGCUGGUCCGUGUCGCCG | SEQ ID NO: 44 |
| Si4-SNP1-C-sense | GGCCGACCAGGCACAGCGG | SEQ ID NO: 45 |
| Si4-SNP1-C-antisense | CCGGCUGGUCCGUGUCGCC | SEQ ID NO: 46 |
| Si5-SNP1-C-sense | CGGCCGACCAGGCACAGCG- | SEQ ID NO: 47 |
| Si5-SNP1-C-antisense | GCCGGCUGGUCCGUGUCGC | SEQ ID NO: 48 |
| Si6-SNP1-C-sense | UCGGCCGACCAGGCACAGC | SEQ ID NO: 49 |
| Si6-SNP1-C-antisense | AGCCGGCUGGUCCGUGUCG | SEQ ID NO: 50 |
| Si7-SNP1-C-sense | GUCGGCCGACCAGGCACAG | SEQ ID NO: 51 |
| Si7-SNP1-C-antisense | CAGCCGGCUGGUCCGUGUC | SEQ ID NO: 52 |
| Si8-SNP1-C-sense | AGUCGGCCGACCAGGCACA | SEQ ID NO: 53 |
| Si8-SNP1-C-antisense | UCAGCCGGCUGGUCCGUGU | SEQ ID NO: 54 |
| Si9-SNP1-C-sense | GAGUCGGCCGACCAGGCAC | SEQ ID NO: 55 |
| Si9-SNP1-C-antisense | CUCAGCCGGCUGGUCCGUG | SEQ ID NO: 56 |
| Si10-SNP1-C-sense | GGAGUCGGCCGACCAGGCA | SEQ ID NO: 57 |
| Si10-SNP1-C-antisense | CCUCAGCCGGCUGGUCCGU | SEQ ID NO: 58 |
| Si11-SNP1-C-sense | AGGAGUCGGCCGACCAGGC | SEQ ID NO: 59 |
| Si11-SNP1-C-antisense | UCCUCAGCCGGCUGGUCCG | SEQ ID NO: 60 |
| Si12-SNP1-C-sense | GAGGAGUCGGCCGACCAGG | SEQ ID NO: 61 |
| Si12-SNP1-C-antisense | CUCCUCAGCCGGCUGGUCC | SEQ ID NO: 62 |
| Si13-SNP1-C-sense | GGAGGAGUCGGCCGACCAG | SEQ ID NO: 63 |
| Si13-SNP1-C-antisense | CCUCCUCAGCCGGCUGGUC | SEQ ID NO: 64 |
| Si14-SNP1-C-sense | UGGAGGAGUCGGCCGACCA | SEQ ID NO: 65 |
| Si14-SNP1-C-antisense | ACCUCCUCAGCCGGCUGGU | SEQ ID NO: 66 |
| Si15-SNP1-C-sense | AUGGAGGAGUCGGCCGACC | SEQ ID NO: 67 |
| Si15-SNP1-C-antisense | UACCUCCUCAGCCGGCUGG | SEQ ID NO: 68 |
| Si16-SNP1-C-sense | CAUGGAGGAGUCGGCCGAC | SEQ ID NO: 69 |
| Si16-SNP1-C-antisense | GUACCUCCUCAGCCGGCUG | SEQ ID NO: 70 |
| Si17-SNP1-C-sense | UCAUGGAGGAGUCGGCCGA | SEQ ID NO: 71 |
| Si17-SNP1-C-antisense | AGUACCUCCUCAGCCGGCU | SEQ ID NO: 72 |
| Si18-SNP1-C-sense | CUCAUGGAGGAGUCGGCCG | SEQ ID NO: 73 |
| Si18-SNP1-C-antisense | GAGUACCUCCUCAGCCGGC | SEQ ID NO: 74 |
| Si19-SNP1-C-sense | CCUCAUGGAGGAGUCGGCC | SEQ ID NO: 75 |
| Si19-SNP1-C-antisense | GGAGUACCUCCUCAGCCGG | SEQ ID NO: 76 |

siRNA against the SNP1 humain (rs2229920) with a polymorphism C. The sequence at the top corresponds to the sense strand (or the sense sequence) (5′-3′) and is call “passenger sequence” and the at the bottom corresponds to the antisense strand (or the antisense sequence) (3′-5′) and is call “guide sequence” (see the Si1-SNP1-C for example).

TABLE 3

| | | Sequence |
|---|---|---|
| Name | siRNA sequence | number |
| Si1-SNP2-A-sense | 5′-AGAACCCUUGACACCAUCC-3′ | SEQ ID NO: 77 |
| Si1-SNP2-A-antisense | 3′-UCUUGGGAACUGUGGUAGG-5′ | SEQ ID NO: 78 |
| Si2-SNP2-A-sense | CAGAACCCUUGACACCAUC | SEQ ID NO: 79 |
| Si2-SNP2-A-antisense | GUCUUGGGAACUGUGGUAG | SEQ ID NO: 80 |
| Si3-SNP2-A-sense | CCAGAACCCUUGACACCAU | SEQ ID NO: 81 |
| Si3-SNP2-A-antisense | GGUCUUGGGAACUGUGGUA | SEQ ID NO: 82 |
| Si4-SNP2-A-sense | ACCAGAACCCUUGACACCA | SEQ ID NO: 83 |
| Si4-SNP2-A-antisense | UGGUCUUGGGAACUGUGGU | SEQ ID NO: 84 |
| Si5-SNP2-A-sense | GACCAGAACCCUUGACACC | SEQ ID NO: 85 |
| Si5-SNP2-A-antisense | CUGGUCUUGGGAACUGUGG | SEQ ID NO: 86 |
| Si6-SNP2-A-sense | GGACCAGAACCCUUGACAC | SEQ ID NO: 87 |
| Si6-SNP2-A-antisense | CCUGGUCUUGGGAACUGUG | SEQ ID NO: 88 |
| Si7-SNP2-A-sense | GGGACCAGAACCCUUGACA | SEQ ID NO: 89 |
| Si7-SNP2-A-antisense | CCCUGGUCUUGGGAACUGU | SEQ ID NO: 90 |
| Si8-SNP2-A-sense | GGGGACCAGAACCCUUGAC | SEQ ID NO: 91 |
| Si8-SNP2-A-antisense | CCCCUGGUCUUGGGAACUG | SEQ ID NO: 92 |
| Si9-SNP2-A-sense | GGGGGACCAGAACCCUUGA | SEQ ID NO: 93 |
| Si9-SNP2-A-antisense | CCCCCUGGUCUUGGGAACU | SEQ ID NO: 94 |
| Si10-SNP2-A-sense | GGGGGGACCAGAACCCUUG | SEQ ID NO: 95 |
| Si10-SNP2-A-antisense | CCCCCCUGGUCUUGGGAAC | SEQ ID NO: 96 |
| Si11-SNP2-A-sense | AGGGGGGACCAGAACCCUU | SEQ ID NO: 97 |
| Si11-SNP2-A-antisense | UCCCCCCUGGUCUUGGGAA | SEQ ID NO: 98 |
| Si12-SNP2-A-sense | GAGGGGGGACCAGAACCCU | SEQ ID NO: 99 |
| Si12-SNP2-A-antisense | CUCCCCCCUGGUCUUGGGA | SEQ ID NO: 100 |
| Si13-SNP2-A-sense | AGAGGGGGGACCAGAACCC | SEQ ID NO: 101 |
| Si13-SNP2-A-antisense | UCUCCCCCCUGGUCUUGGG | SEQ ID NO: 102 |
| Si14-SNP2-A-sense | CAGAGGGGGGACCAGAACC | SEQ ID NO: 103 |
| Si14-SNP2-A-antisense | GUCUCCCCCCUGGUCUUGG | SEQ ID NO: 104 |
| Si15-SNP2-A-sense | GCAGAGGGGGGACCAGAAC | SEQ ID NO: 105 |
| Si15-SNP2-A-antisense | CGUCUCCCCCCUGGUCUUG | SEQ ID NO: 106 |
| Si16-SNP2-A-sense | GGCAGAGGGGGGACCAGAA | SEQ ID NO: 107 |
| Si16-SNP2-A-antisense | CCGUCUCCCCCCUGGUCUU | SEQ ID NO: 108 |
| Si17-SNP2-A-sense | UGGCAGAGGGGGGACCAGA | SEQ ID NO: 109 |
| Si17-SNP2-A-antisense | ACCGUCUCCCCCCUGGUCU | SEQ ID NO: 110 |
| Si18-SNP2-A-sense | GUGGCAGAGGGGGGACCAG | SEQ ID NO: 111 |
| Si18-SNP2-A-antisense | CACCGUCUCCCCCCUGGUC | SEQ ID NO: 112 |
| Si19-SNP2-A-sense | GGUGGCAGAGGGGGGACCA | SEQ ID NO: 113 |
| Si19-SNP2-A-antisense | CCACCGUCUCCCCCCUGGU | SEQ ID NO: 114 |

siRNA against the SNP2 humain (rs12461992) with a polymorphism A. The sequence at the top corresponds to the sense strand (or the sense sequence) (5′-3′) and is call “passenger sequence” and the at the bottom corresponds to the antisense strand (or the antisense sequence) (3′-5′) and is call “guide sequence” (see the Si1-SNP2-A for example).

TABLE 4

| | | Sequence |
|---|---|---|
| Name | siRNA sequence | number |
| Si1-SNP2-T-sense | 5′-UGAACCCUUGACACCAUCC-3′ | SEQ ID NO: 115 |
| Si1-SNP2-T-antisense | 3′-ACUUGGGAACUGUGGUAGG-5′ | SEQ ID NO: 116 |
| Si2-SNP2-T-sense | CUGAACCCUUGACACCAUC | SEQ ID NO: 117 |
| Si2-SNP2-T-antisense | GACUUGGGAACUGUGGUAG | SEQ ID NO: 118 |
| Si3-SNP2-T-sense | CCUGAACCCUUGACACCAU | SEQ ID NO: 119 |
| Si3-SNP2-T-antisense | GGACUUGGGAACUGUGGUA | SEQ ID NO: 120 |
| Si4-SNP2-T-sense | ACCUGAACCCUUGACACCA | SEQ ID NO: 121 |
| Si4-SNP2-T-antisense | UGGACUUGGGAACUGUGGU | SEQ ID NO: 122 |
| Si5-SNP2-T-sense | GACCUGAACCCUUGACACC | SEQ ID NO: 123 |
| Si5-SNP2-T-antisense | CUGGACUUGGGAACUGUGG | SEQ ID NO: 124 |
| Si6-SNP2-T-sense | GGACCUGAACCCUUGACAC | SEQ ID NO: 125 |
| Si6-SNP2-T-antisense | CCUGGACUUGGGAACUGUG | SEQ ID NO: 126 |
| Si7-SNP2-T-sense | GGGACCUGAACCCUUGACA | SEQ ID NO: 127 |
| Si7-SNP2-T-antisense | CCCUGGACUUGGGAACUGU | SEQ ID NO: 128 |
| Si8-SNP2-T-sense | GGGGACCUGAACCCUUGAC | SEQ ID NO: 129 |
| Si8-SNP2-T-antisense | CCCCUGGACUUGGGAACUG | SEQ ID NO: 130 |
| Si9-SNP2-T-sense | GGGGGACCUGAACCCUUGA | SEQ ID NO: 131 |
| Si9-SNP2-T-antisense | CCCCCUGGACUUGGGAACU | SEQ ID NO: 132 |
| Si10-SNP2-T-sense | GGGGGGACCUGAACCCUUG | SEQ ID NO: 133 |
| Si10-SNP2-T-antisense | CCCCCCUGGACUUGGGAAC | SEQ ID NO: 134 |
| Si11-SNP2-T-sense | AGGGGGGACCUGAACCCUU | SEQ ID NO: 135 |
| Si11-SNP2-T-antisense | UCCCCCCUGGACUUGGGAA | SEQ ID NO: 136 |
| Si12-SNP2-T-sense | GAGGGGGGACCUGAACCCU | SEQ ID NO: 137 |
| Si12-SNP2-T-antisense | CUCCCCCCUGGACUUGGGA | SEQ ID NO: 138 |
| Si13-SNP2-T-sense | AGAGGGGGGACCUGAACCC | SEQ ID NO: 139 |
| Si13-SNP2-T-antisense | UCUCCCCCCUGGACUUGGG | SEQ ID NO: 140 |
| Si14-SNP2-T-sense | CAGAGGGGGGACCUGAACC | SEQ ID NO: 141 |
| Si14-SNP2-T-antisense | GUCUCCCCCCUGGACUUGG | SEQ ID NO: 142 |
| Si15-SNP2-T-sense | GCAGAGGGGGGACCUGAAC | SEQ ID NO: 143 |
| Si15-SNP2-T-antisense | CGUCUCCCCCCUGGACUUG | SEQ ID NO: 144 |
| Si16-SNP2-T-sense | GGCAGAGGGGGGACCUGAA | SEQ ID NO: 145 |
| Si16-SNP2-T-antisense | CCGUCUCCCCCCUGGACUU | SEQ ID NO: 146 |
| Si17-SNP2-T-sense | UGGCAGAGGGGGGACCUGA | SEQ ID NO: 147 |
| Si17-SNP2-T-antisense | ACCGUCUCCCCCCUGGACU | SEQ ID NO: 148 |
| Si18-SNP2-T-sense | GUGGCAGAGGGGGGACCUG | SEQ ID NO: 149 |
| Si18-SNP2-T-antisense | CACCGUCUCCCCCCUGGAC | SEQ ID NO: 150 |
| Si19-SNP2-T-sense | GGUGGCAGAGGGGGGACCU | SEQ ID NO: 151 |
| Si19-SNP2-T-antisense | CCACCGUCUCCCCCCUGGA | SEQ ID NO: 152 |

siRNA against the SNP2 humain (rs12461992) with a polymorphism T. The sequence at the top corresponds to the sense strand (or the sense sequence) (5′-3′) and is call “passenger sequence” and the at the bottom corresponds to the antisense strand (or the antisense sequence) (3′-5′) and is call “guide sequence” (see the Si1-SNP2-T for example).

The AS-siRNA of the invention is able to silence only one allele of DNM2 gene comprising a heterozygous non-pathological polymorphism, by hybridizing specifically to the gene transcript (messenger RNA or mRNA) derived from said allele of DNM2 gene. AS-siRNA of the invention is therefore complementary to mRNA derived from said allele of DNM2 and binds to said mRNA by base pairing. The term “complementary” refers to the ability of polynucleotides to form base pairs with another polynucleotide molecule. Base pairs are typically formed by hydrogen bonds between nucleotide units in antiparallel polynucleotide strands. Particularly, the degree of complementarity between the AS-siRNA according to the invention and the target mRNA is equal to about 100%.

In a particular embodiment, AS-siRNA of the invention targets a region of the DNM2 gene transcript comprising said non-pathological polymorphism. Accordingly, the AS-siRNA of the invention is complementary to a sequence of the mRNA comprising said non-pathological polymorphism. The specificity of siRNA allows discriminating two sequences, even when differing by a single nucleotide. This property allows the AS-siRNA of the invention targeting polymorphism resulting to single nucleotide substitution.

In one particular embodiment, the target allele (and consequently the target mRNA) could be arbitrarily chosen, in absence of disease-causing mutation, in order to reduce the level of DNM2 mRNA and/or protein. In particular, the AS-siRNA of the invention could target the mRNA carrying or not said non-pathological polymorphism, when the goal is only to reduce the overall level of DNM2 mRNA or DNM2 protein. For example, the AS-siRNA of the invention could target anyone of the DNM2 allele carrying one of the two version of the heterozygous non-pathological polymorphism, wherein DNM2 is overexpressed in a cell, for example in X-linked myotubular myopathy or cancer such as prostate cancer and pancreatic cancer.

In another particular embodiment, the targeted version of the non-pathological polymorphism is present on the same allele as a disease-causing mutation. Therefore, the AS-siRNA of the invention targets and silences only the allele carrying said targeted version of the polymorphism and said disease-causing mutation. This particular embodiment requires prior confirming the location of the disease-causing mutation.

### AS-siRNA Targeting the Allele of the DNM2 Gene Comprising a Disease-Causing Mutation

In one embodiment of the invention, the DNM2 gene is heterozygous for the presence of a disease-causing mutation. Therefore, the DNM2 gene is present in two different forms corresponding to the two alleles: one DNM2 allele is a “wild type allele” whereas the other is a “mutant allele”. In this embodiment, the AS-siRNA of the invention specifically targets and silences the allele of the DNM2 gene comprising said disease-causing mutation without targeting the wild type allele. The AS-siRNA is thus able to silence the expression of Dynamin 2 mRNA and Dynamin 2 protein derived from the mutant allele without affecting the expression of mRNA and protein derived from the wild type allele.

In particular, the disease-causing mutation could be any deletion, insertion or substitution of nucleotide(s) within the DNM2 gene which is responsible for a pathology or which is correlated to a pathology. In a preferred embodiment, the disease-causing mutation is a dominant mutation. By dominant mutation is meant any mutation that leads to a dominant allele. By “dominant allele” is meant an allele that exerts its effect on phenotype over the presence of a recessive allele of the same gene. The terms dominant and recessive alleles are defined relative to one another and are not absolute. In other words, the phenotypic consequences of a dominant mutation are observed in a heterozygous individual carrying one mutant allele and one wild type allele. Recessive alleles only show their effect if the individual has two copies of the mutated allele (also known as being homozygous) or two different mutated alleles (also known as composite heterozygosity).

In a particular embodiment, the dominant mutation is a gain-of-function mutation. A gain-of-function mutation is defined as a mutation that confers new or enhanced activity on a protein. A gain-of-function mutation is a type of mutation in which the altered gene product possesses a new molecular function or a new pattern of gene expression. Consequently, the disease-causing mutation within the DNM2 leads to a gain-of-function of Dynamin 2 protein.

In a particular embodiment, the dominant mutation is a loss-of-function mutation by dominant negative effect. A loss-of-function mutation is defined as a mutation that results in the loss or reduction of the normal activity of a protein. Dominant-negative effect is defined as the product of a mutated allele alters the function of the product from the wild-type allele. This occurs, for example, when oligomerization is required for normal function on a protein and when the mutated protein is able to oligomerize with the wild-type protein. Consequently, the disease-causing mutation within the DNM2 leads to a loss-of-function of the wild-type Dynamin 2 protein due to the presence of the mutated Dynamin 2 protein.

In another particular embodiment, the DNM2 gene which is heterozygous for a disease-causing mutation is not haploinsufficient. Haploinsufficiency occurs when one copy of a gene is inactivated or deleted and the remaining functional copy of the gene is not adequate to produce sufficient amount of the gene product to preserve normal function. In other words, the wild type DNM2 allele of the invention is able to preserve normal function, following the silencing of the mutant allele by AS-siRNA of the invention. Therefore, the present invention preferably relates to autosomal dominant disease in which there is no haploinsufficiency.

In a particular embodiment, the dominant mutation within the DNM2 gene leads to an autosomal dominant disease. An autosomal dominant disease is a disease wherein the individual has one copy of a mutant gene and one normal gene on a pair of autosomal chromosomes (autosomal chromosome being any chromosome which is not a sex chromosome). An individual with autosomal dominant diseases has 50% chance of passing the mutant gene and therefore the disorder on to each of its children.

In one preferred embodiment, the autosomal dominant disease is selected from Autosomal Dominant Centronuclear Myopathy (AD-CNM), T-cell acute lymphoblastic leukemia, Charcot-Marie-Tooth disease (CMT) or Hereditary Spastic Paraplegia (HSP). Preferably, the autosomal dominant disease is Autosomal Dominant Centronuclear Myopathy (AD-CNM).

In another embodiment, the disease-causing mutation within the DNM2 gene is responsible for or correlated to a disease selected from Autosomal Dominant Centronuclear Myopathy (AD-CNM), T-cell acute lymphoblastic leukemia, Charcot-Marie-Tooth disease (CMT) or Hereditary Spastic Paraplegia (HSP). Preferably, the disease-causing mutation within the DNM2 gene is responsible for or correlated to Autosomal Dominant Centronuclear Myopathy (AD-CNM).

According to the invention, the disease-causing mutations c.1393C>T and c.1856C>T are respectively responsible for the following substitution in the DNM2 protein sequence: p.R465W and p.S619L.

Thus, in a particular aspect, the invention relates to an AS-siRNA able to silence the expression of only one allele of a heterozygous DNM2 gene in a cell, wherein the targeted allele comprises a disease-causing mutation selected from the group consisting of c.1393C>T or c.1856C>T.

The inventors have indeed designed many siRNA able to target the most frequent disease-causing mutation (c.1856C>T also called S619L and c.1393C>T also called p.R465W) and tested in in-vitro and in-vivo experiments. These sequences are listed in the tables 5 to 6.

Thus, the invention also relates to an allele specific siRNA (AS-siRNA) able to silence the expression of only one allele of a heterozygous DNM2 gene wherein the AS-siRNA comprises a sense strand selected in the group consisting of SEQ ID NO: 153, 155, 157, 159, 161, 163, 165, 167, 169, 171, 173, 175, 177, 179, 181, 183, 185, 187, 189, 191, 193, 195, 197, 199, 201, 203, 205, 215, 217, 223, 225 and 227.

Particularly, the invention relates to an allele specific siRNA (AS-siRNA) able to silence the expression of only one allele of a heterozygous DNM2 gene wherein the AS-siRNA comprises a sense strand selected in the group consisting of SEQ ID NO: 157, 163, 167, 171, 175, 177, 179, 181 and 185.

TABLE 5

| | | Sequence |
|---|---|---|
| Name | siRNA sequence | number |
| Si1-S619L-sense | 5′-UGUUCCUCCGAGCUGGCGU-3′ | SEQ ID NO: 153 |
| Si1-S619L-antisense | 3′-ACAAGGAGGCUCGACCGCA-5′ | SEQ ID NO: 154 |
| Si2-S619L-sense | UUGUUCCUCCGAGCUGGCG | SEQ ID NO: 155 |
| Si2-S619L-antisense | AACAAGGAGGCUCGACCGC | SEQ ID NO: 156 |
| Si3-S619L-sense | CUUGUUCCUCCGAGCUGGC | SEQ ID NO: 157 |
| Si3-S619L-antisense | GAACAAGGAGGCUCGACCG | SEQ ID NO: 158 |
| Si4-S619L-sense | CCUUGUUCCUCCGAGCUGG | SEQ ID NO: 159 |
| Si4-S619L-antisense | GGAACAAGGAGGCUCGACC | SEQ ID NO: 160 |
| Si5-S619L-sense | GCCUUGUUCCUCCGAGCUG | SEQ ID NO: 161 |
| Si5-S619L-antisense | CGGAACAAGGAGGCUCGAC | SEQ ID NO: 162 |
| Si6-S619L-sense | GGCCUUGUUCCUCCGAGCU | SEQ ID NO: 163 |
| Si6-S619L-antisense | CCGGAACAAGGAGGCUCGA | SEQ ID NO: 164 |
| Si7-S619L-sense | AGGCCUUGUUCCUCCGAGC | SEQ ID NO: 165 |
| Si7-S619L-antisense | UCCGGAACAAGGAGGCUCG | SEQ ID NO: 166 |
| Si8-S619L-sense | AAGGCCUUGUUCCUCCGAG | SEQ ID NO: 167 |
| Si8-S619L-antisense | UUCCGGAACAAGGAGGCUC | SEQ ID NO: 168 |
| Si9-S619L-sense | GAAGGCCUUGUUCCUCCGA | SEQ ID NO: 169 |
| Si9-S619L-antisense | CUUCCGGAACAAGGAGGCU | SEQ ID NO: 170 |
| Si10-S619L-sense | GGAAGGCCUUGUUCCUCCG | SEQ ID NO: 171 |
| Si10-S619L-antisense | CCUUCCGGAACAAGGAGGC | SEQ ID NO: 172 |
| Si11-S619L-sense | UGGAAGGCCUUGUUCCUCC | SEQ ID NO: 173 |
| Si11-S619L-antisense | ACCUUCCGGAACAAGGAGG | SEQ ID NO: 174 |
| Si12-S619L-sense | CUGGAAGGCCUUGUUCCUC | SEQ ID NO: 175 |
| Si12-S619L-antisense | GACCUUCCGGAACAAGGAG | SEQ ID NO: 176 |
| Si13-S619L-sense | GCUGGAAGGCCUUGUUCCU | SEQ ID NO: 177 |
| Si13-S619L-antisense | CGACCUUCCGGAACAAGGA | SEQ ID NO: 178 |
| Si14-S619L-sense | AGCUGGAAGGCCUUGUUCC | SEQ ID NO: 179 |
| Si14-S619L-antisense | UCGACCUUCCGGAACAAGG | SEQ ID NO: 180 |
| Si15-S619L-sense | CAGCUGGAAGGCCUUGUUC | SEQ ID NO: 181 |
| Si15-S619L-antisense | GUCGACCUUCCGGAACAAG | SEQ ID NO: 182 |
| Si16-S619L-sense | ACAGCUGGAAGGCCUUGUU | SEQ ID NO: 183 |
| Si16-S619L-antisense | UGUCGACCUUCCGGAACAA | SEQ ID NO: 184 |
| Si17-S619L-sense | GACAGCUGGAAGGCCUUGU | SEQ ID NO: 185 |
| Si17-S619L-antisense | CUGUCGACCUUCCGGAACA | SEQ ID NO: 186 |
| Si18-S619L-sense | GGACAGCUGGAAGGCCUUG | SEQ ID NO: 187 |
| Si18-S619L-antisense | CCUGUCGACCUUCCGGAAC | SEQ ID NO: 188 |
| Si19-S619L-sense | UGGACAGCUGGAAGGCCUU | SEQ ID NO: 189 |
| Si19-S619L-antisense | ACCUGUCGACCUUCCGGAA | SEQ ID NO: 190 |

siRNA against the mutation S619L. The sequence at the top corresponds to the sense strand (or the sense sequence) (5′-3′) and is call “passenger sequence” and the at the bottom corresponds to the antisense strand (or the antisense sequence) (3′-5′) and is call “guide sequence” (see the Si1-S619L for example).

TABLE 6

| | | Sequence |
|---|---|---|
| Name | siRNA sequence | number |
| Si1-R465W-sense | 5′-UGGGAACGGGAGGGGAGAA-3′ | SEQ ID NO: 191 |
| Si1-R465W-antisense | 3′-ACCCUUGCCCUCCCCUCUU-5′ | SEQ ID NO: 192 |
| Si2-R465W-sense | CUGGGAACGGGAGGGGAGA | SEQ ID NO: 193 |
| Si2-R465W-antisense | GACCCUUGCCCUCCCCUCU | SEQ ID NO: 194 |
| Si3-R465W-sense | UCUGGGAACGGGAGGGGAG | SEQ ID NO: 195 |
| Si3-R465W-antisense | AGACCCUUGCCCUCCCCUC | SEQ ID NO: 196 |
| Si4-R465W-sense | AUCUGGGAACGGGAGGGGA | SEQ ID NO: 197 |
| Si4-R465W-antisense | UAGACCCUUGCCCUCCCCU | SEQ ID NO: 198 |
| Si5-R465W-sense | CAUCUGGGAACGGGAGGGG | SEQ ID NO: 199 |
| Si5-R465W-antisense | GUAGACCCUUGCCCUCCCC | SEQ ID NO: 200 |
| Si6-R465W-sense | ACAUCUGGGAACGGGAGGG | SEQ ID NO: 201 |
| Si6-R465W-antisense | UGUAGACCCUUGCCCUCCC | SEQ ID NO: 202 |
| Si7-R465W-sense | UACAUCUGGGAACGGGAGG | SEQ ID NO: 203 |
| Si7-R465W-antisense | AUGUAGACCCUUGCCCUCC | SEQ ID NO: 204 |
| Si8-R465W-sense | UUACAUCUGGGAACGGGAG | SEQ ID NO: 205 |
| Si8-R465W-antisense | AAUGUAGACCCUUGCCCUC | SEQ ID NO: 206 |
| Si9-R465W-sense | CUUACAUCUGGGAACGGGA | SEQ ID NO: 207 |
| Si9-R465W-antisense | GAAUGUAGACCCUUGCCCU | SEQ ID NO: 208 |
| Si10-R465W-sense | ACUUACAUCUGGGAACGGG | SEQ ID NO: 209 |
| Si10-R465W-antisense | UGAAUGUAGACCCUUGCCC | SEQ ID NO: 210 |
| Si11-R465W-sense | CACUUACAUCUGGGAACGG | SEQ ID NO: 211 |
| Si11-R465W-antisense | GUGAAUGUAGACCCUUGCC | SEQ ID NO: 212 |
| Si12-R465W-sense | CCACUUACAUCUGGGAACG | SEQ ID NO: 213 |
| Si12-R465W-antisense | GGUGAAUGUAGACCCUUGC | SEQ ID NO: 214 |
| Si13-R465W-sense | ACCACUUACAUCUGGGAAC | SEQ ID NO: 215 |
| Si13-R465W-antisense | UGGUGAAUGUAGACCCUUG | SEQ ID NO: 216 |
| Si14-R465W-sense | CACCACUUACAUCUGGGAA | SEQ ID NO: 217 |
| Si14-R465W-antisense | GUGGUGAAUGUAGACCCUU | SEQ ID NO: 218 |
| Si15-R465W-sense | UCACCACUUACAUCUGGGA | SEQ ID NO: 219 |
| Si15-R465W-antisense | AGUGGUGAAUGUAGACCCU | SEQ ID NO: 220 |
| Si16-R465W-sense | GUCACCACUUACAUCUGGG | SEQ ID NO: 221 |
| Si16-R465W-antisense | CAGUGGUGAAUGUAGACCC | SEQ ID NO: 222 |
| Si17-R465W-sense | CGUCACCACUUACAUCUGG | SEQ ID NO: 223 |
| Si17-R465W-antisense | GCAGUGGUGAAUGUAGACC | SEQ ID NO: 224 |
| Si18-R465W-sense | UCGUCACCACUUACAUCUG | SEQ ID NO: 225 |
| Si18-R465W-antisense | AGCAGUGGUGAAUGUAGAC | SEQ ID NO: 226 |
| Si19-R465W-sense | AUCGUCACCACUUACAUCU | SEQ ID NO: 227 |
| Si19-R465W-antisense | UAGCAGUGGUGAAUGUAGA | SEQ ID NO: 228 |

siRNA against the mutation R465W. The sequence at the top corresponds to the sense strand (or the antisense sequence) (5′-3′) and is call “passenger sequence” and the at the bottom corresponds to the antisense strand (or the antisense sequence) (3′-5′) and is call “guide sequence” (see the Si1-R465W for example).

The AS-siRNA of the invention is able to silence the mutant allele of DNM2 gene, by hybridizing specifically to the gene transcript (messenger RNA or mRNA) derived from said mutant allele of DNM2 gene. The AS-siRNA of the invention is therefore complementary to a mRNA derived from said mutant allele of DNM2 and binds to said mRNA by base pairing. The term “complementary” refers to the ability of polynucleotides to form base pairs with another polynucleotide molecule. Base pairs are typically formed by hydrogen bonds between nucleotide units in antiparallel polynucleotide strands. Preferably, the degree of complementarity between the AS-siRNA according to the invention and the target mRNA is equal to about 100%.

In a particular embodiment, the AS-siRNA of the invention targets a region of the DNM2 gene transcript comprising said disease-causing mutation. Accordingly, the AS-siRNA of the invention is complementary to a sequence of the mRNA comprising said disease-causing mutation. The specificity of siRNA allows discriminating two sequences, even when differing by a single nucleotide. This property allows the AS-siRNA of the invention targeting disease-causing mutations, such as mutations resulting to single nucleotide substitution.

General Information on all siRNAs of the Invention

In a particular embodiment, the AS-siRNA of the invention contains nucleotide overhangs on 3′ end of each strand. In a more particular embodiment, the AS-siRNA of the invention contains dinucleotide overhangs made of two deoxythymidines (dTdT) on 3′ end of each strand (sense and antisense).

Another aspect of the invention relates to a vector encoding the AS-siRNA of the invention, the vector being particularly a plasmid or a viral vector, such as an AAV vector.

By “sense strand” is meant the strand of the AS-siRNA which has the same sequence as the targeted allele comprising the non-pathological polymorphism or the disease-causing mutation. Therefore, the other strand of AS-siRNA is called “anti-sense” because its sequence is complementary to the targeted DNM2 mRNA, which is called the “sense” sequence (so that a sense segment of mRNA “5′-AAGGUC-3′” would be blocked by the anti-sense mRNA segment “3′-UUCCAG-5′”).

In a particular embodiment, the AS-siRNA of the invention is used to reduce expression of DNM2 mRNA and/or DNM2 protein by 20-60%, such as 20, 30, 40, 50 or 60%. Particularly, the AS-siRNA of the invention is used to reduce expression of DNM2 mRNA and/or DNM2 protein by about 50%. By «about» is meant a value of + or −10%, Particularly + or −5%. For example, about 50% means from 45 to 55%, Particularly from 47.5 to 52.5%.

The present invention contemplates various ways of reaching the target mRNA with AS-siRNA of the invention. The AS-siRNA may be administered to the cell as isolated oligonucleotide, either directly or using transfection reagents such as lipidic derivatives, liposomes, calcium phosphate, nanoparticles, microinjection or electroporation.

In another embodiment, the present invention contemplates introducing the AS-siRNA into the cell in the form of a vector. Thus, another aspect of the present invention relates to a vector encoding the AS-siRNA of the invention. The vector may in particular be a plasmid or a viral vector. Representative viral vectors useful in the practice of the invention include, without limitation, a vector derived from adenovirus, retrovirus, in particular lentivirus, poxviruses, herpes simplex virus I and adeno-associated virus (AAV).

In a particular embodiment, the AAV vector is AAV1, AAV2, AAV3, AAV4, AA5, AAV6, AAV7, AAV8, AAV9 AAV10, AAV3B, AAV-218, Rh10, Rh74 or any other serotypes of AAV that can infect human, monkeys or other species.

Selection of the appropriate viral vector will of course depend on the targeted cell and the virus tropism. In a particular embodiment, targeted cells are muscle cells, but viral vectors with broad tropism, including in particular the muscle tropism, may also be implemented. In a particular embodiment, an AAV1 vector is implemented, for example for use in intramuscular injections. In another embodiment, the vector is to be administered via the systemic route (for example via the intravascular or intraarterial route), and the vector is an AAV8 or AAV9 vector.

In a particular embodiment, the invention also relates to shRNA (short hairpin RNA) corresponding to the AS-siRNA of the invention, with a further tight hairpin turn. The shRNA hairpin structure is then cleaved by the cellular machinery into siRNA. A further aspect of the invention relates to a vector encoding shRNA corresponding to AS-siRNA of the invention.

In another aspect, the invention also relates to a target cell comprising an AS-siRNA of the invention or which is transfected or transduced with a vector of the invention. For example, the target cell may be selected from: a muscle cell (or a cell of the muscle lineage), such as myoblast, for example a patient-derived myoblast, or a fibroblast such as a patient-derived fibroblast.

### Uses of the AS-siRNA of the Invention

In another aspect, the present invention relates to an in vitro method for silencing the expression of the mutated allele of DNM2 gene without silencing the expression of the wild type allele of the DNM2 gene in a target cell, such as a muscle target cell (for example a muscle cell, such as a myoblast, in particular a patient-derived myoblast), comprising introducing in said target cell an AS-siRNA or a vector of the invention.

In another aspect, the present invention relates to an in vitro method for silencing the expression of one allele of DNM2 gene carrying a heterozygous non-pathological polymorphism without silencing the expression of the other allele of the DNM2 gene in a target cell, comprising introducing in said target cell an AS-siRNA or a vector of the invention.

In a further aspect, the present invention relates to an AS-siRNA, a vector or a cell of the invention for use in a method for treating, in a subject in need thereof, a disease induced by a disease-causing mutation in the DNM2 gene. Particularly, an AS-siRNA, a vector or a cell of the invention are used in a method for treating, in a subject in need thereof, a centronuclear myopathy (such as autosomal dominant centronuclear myopathy), T-cell acute lymphoblastic leukemia, Charcot-Marie-Tooth disease (CMT) or Hereditary Spastic Paraplegia (HSP). More preferably, an AS-siRNA, a vector or a cell of the invention are used in a method for treating autosomal dominant centronuclear myopathy.

In another aspect, the present invention relates to an AS-siRNA, a vector or a cell of the invention for use in a method for treating, in a subject in need thereof a muscular dystrophy such as Duchenne muscular dystrophy.

In another aspect, the present invention relates to an AS-siRNA, a vector or a cell of the invention for use in a method for treating, in subject in need thereof, a disease associated with overexpression of dynamin 2, preferably for treating X-linked myotubular myopathy, or cancer such as prostate cancer and pancreatic cancer.

In a particular embodiment, the invention relates to an AS-siRNA, a vector or a cell of the invention for use in the treatment of a centronuclear myopathy (such as autosomal dominant centronuclear myopathy), T-cell acute lymphoblastic leukemia, Charcot-Marie-Tooth disease (CMT) or Hereditary Spastic Paraplegia (HSP) in a subject in need thereof.

In a particular embodiment, the invention relates to a method for treating muscular dystrophy such as Duchenne muscular dystrophy in a subject in need thereof comprising administrating to said subject an effective amount of an AS-siRNA, vector or cell of the invention.

As used herein, the term “subject” denotes a mammal, such as a rodent, a feline, a canine, and a primate. Particularly, the subject according to the invention is a human.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The AS-siRNA of the invention, the vector or the cell according to the invention can be formulated and administered to treat any disease caused by a heterozygous mutation in the DNM2 gene or caused by overexpression of DNM2, preferably to treat autosomal dominant centronuclear myopathy, T-cell acute lymphoblastic leukemia, Charcot-Marie-Tooth disease, Hereditary Spastic Paraplegia, X-linked myotubular myopathy, or cancer such as prostate cancer and pancreatic cancer. AS-siRNA of the invention, the vector or the cell according to the invention are formulated by any means that produces contact of the AS-siRNA with its site of action in the subject in need thereof.

The present invention also provides pharmaceutical compositions comprising the AS-siRNA of the invention, the vector or the cell according to the invention. Such compositions comprise a therapeutically effective amount of the therapeutic (the AS-siRNA, vector or cell of the invention), and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as saline solution, water and oils, including those of petroleum, animal, vegetable or synthetic origin, such as peanut oil, soybean oil, mineral oil, sesame oil and the like. Physiological saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject.

The pharmaceutical composition is adapted for any type of administration to a mammal, in particular a human being and is formulated in accordance with routine procedures. The composition is formulated by using suitable conventional pharmaceutical carrier, diluent and/or excipient. Administration of the composition may be via any common route so long as the target tissue is available via that route.

The amount of the therapeutic of the invention which will be effective in the treatment of a nucleotide repeat expansion can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage of the AS-siRNA, the vector or the cell administered to the subject in need thereof will vary based on several factors including, without limitation, the route of administration, the subject's age or the level of expression necessary to obtain the required therapeutic effect. One skilled in the art can readily determine, based on its knowledge in this field, the dosage range required based on these factors and others.

The invention will be further illustrated by the following figures and examples. However, these examples and figures should not be interpreted in any way as limiting the scope of the present invention.

### FIGURES

FIG. 1: Identification of allele-specific siRNA against the C version of SNP1. A. Expression of the DNM2 mRNA 48 hours after siRNA transfection at 30 nM. Quantification of DNM2 expression normalized to HPRT (n≥4 per condition). B. Quantification of the C/T ratio (n≥4 per condition) after transfection with siRNA at 30 nM. C. Expression of the DNM2 mRNA 48 hours after siRNA transfection at 100 nM. Quantification of DNM2 expression normalized to HPRT (n≥4 per condition). D. Quantification of the C/T ratio (n≥4 per condition) after transfection with siRNA at 100 nM. E. Quantification of the signal by densitometry after siRNA transfection at 100 nM concentration. GAPDH was used as a loading control (n≥4). Data information: In scatter plots, the bars are mean values and error bars indicate SEM. ****P<0.0001, ***P<0.001, **P<0.01 and *P<0.5 using a two-tailed Mann-Whitney U-test relative to the scramble siRNA (Sc).

FIG. 2: Identification of allele-specific siRNA against the T version of SNP1. A. Expression of the DNM2 mRNA 48 hours after siRNA transfection at 30 nM. Quantification of DNM2 expression normalized to HPRT (n≥4 per condition). B. Quantification of the T/C ratio (n≥4 per condition) after transfection with siRNA at 30 nM. C. Expression of the DNM2 mRNA 48 hours after siRNA transfection at 100 nM. Quantification of DNM2 expression normalized to HPRT (n≥4 per condition). D. Quantification of the T/C ratio (n≥4 per condition) after transfection with siRNA at 100 nM. E. Quantification of the signal by densitometry after siRNA transfection at 100 nM concentration. GAPDH was used as a loading control (n≥4). Data information: In scatter plots, the bars are mean values and error bars indicate SEM. *P<0.5 using a two-tailed Mann-Whitney U-test relative to the scramble siRNA (Sc).

FIG. 3: Identification of allele-specific siRNA against the T version of SNP2. A. Expression of the DNM2 mRNA 48 hours after siRNA transfection at 30 nM. Quantification of DNM2 expression normalized to HPRT (n≥4 per condition). B. Quantification of the T/A ratio (n≥4 per condition) after transfection with siRNA at 30 nM. C. Expression of the DNM2 mRNA 48 hours after siRNA transfection at 100 nM. Quantification of DNM2 expression normalized to HPRT (n≥4 per condition). D. Quantification of the T/A ratio (n≥4 per condition) after transfection with siRNA at 100 nM. E. Quantification of the signal by densitometry after siRNA transfection at 100 nM concentration. GAPDH was used as a loading control (n≥4). Data information: In scatter plots, the bars are mean values and error bars indicate SEM. ****P<0.0001, ***P<0.001, **P<0.01 and *P<0.5 using a two-tailed Mann-Whitney U-test relative to the scramble siRNA (Sc).

FIG. 4: Identification of allele-specific siRNA against the A version of SNP2. A. Expression of the DNM2 mRNA 48 hours after siRNA transfection at 30 nM. Quantification of DNM2 expression normalized to HPRT (n≥4 per condition). B. Quantification of the A/T ratio (n≥4 per condition) after transfection with siRNA at 30 nM. C. Expression of the DNM2 mRNA 48 hours after siRNA transfection at 100 nM. Quantification of DNM2 expression normalized to HPRT (n≥4 per condition). D. Quantification of the A/T ratio (n≥4 per condition) after transfection with siRNA at 100 nM. E. Quantification of the signal by densitometry after siRNA transfection at 100 nM concentration. GAPDH was used as a loading control (n≥4). Data information: In scatter plots, the bars are mean values and error bars indicate SEM. **P<0.01 and *P<0.5 using a two-tailed Mann-Whitney U-test relative to the scramble siRNA (Sc).

FIG. 5: Identification of allele-specific siRNA against the S619L DNM2 mutation. A. Expression of the DNM2 mRNA 48 hours after siRNA transfection at 30 nM. Quantification of DNM2 expression normalized to HPRT (n≥4 per condition). B. Quantification of the mutant/WT ratio (n≥4 per condition) after transfection with siRNA at 30 nM. The mutant allele harbors the C version of SNP1. C. Expression of the DNM2 mRNA 48 hours after siRNA transfection at 100 nM. Quantification of DNM2 expression normalized to HPRT (n≥4 per condition). D. Quantification of the mutant/WT ratio (n≥4 per condition) after transfection with siRNA at 100 nM. E. Quantification of the signal by densitometry after siRNA transfection at 100 nM concentration. GAPDH was used as a loading control (n≥4). Data information: In scatter plots, the bars are mean values and error bars indicate SEM. ***P<0.001 and *P<0.1 using a two-tailed Mann-Whitney U-test relative to the scramble siRNA (Sc).

FIG. 6: Impact of AS-siRNA on transferrin uptake and cell surface of patient-derived fibroblasts. A. Transferrin uptake under basal conditions (n=500-700 cells from 3 independent experiments). CTCF: Corrected total cell fluorescence. B. Transferrin uptake after 48 hours transfection with 30 nM scramble siRNA (sc) or allele-specific siRNA against the S619L mutation or the 2 SNPs (n=200-300 cells from 2 independent experiments). C. Transferrin uptake after 48 hours transfection with 100 nM scramble siRNA (sc) or allele-specific siRNA against the S619L mutation or the 2 SNPs (n=200-300 cells from 2 independent experiments). D. Cell surface (μm2) under basal conditions (n=500-700 cells from 3 independent experiments). E. Cell surface after 48 hours transfection with 30 nM scramble siRNA (sc) or allele-specific siRNA against the S619L mutation or the 2 SNPs (n=200-300 cells from 2 independent experiments). F. Cell surface after 48 hours transfection with 100 nM scramble siRNA (sc) or allele-specific siRNA against the S619L mutation or the 2 SNPs (n=200-300 cells from 2 independent experiments). Data information: bars represent mean values and error bars indicate SEM. Statistical analysis are performed using Kruskal-Wallis for A to F (**** p<0.0001 in all histograms), followed by Dunn's test relative to control cells in A and D (**** P<0.0001, *** P<0.001 adjusted p-value), control cells transfected with scramble siRNA in B, C, E and F (**** P<0.0001 adjusted p-values), or the patient cell lines transfected with scramble siRNA in B, C, E and F (†P<0.05, ††P<0.01, and ††††P<0.0001 adjusted p-values).

FIG. 7: Migration and adhesion assays in patient-derived cells. A. Mean speed of CNM- and control-fibroblasts (μm/minute) under basal conditions (n=50 cells tracked). B. Mean speed of cells after 48 hours transfection with 30 nM scramble siRNA (sc) or allele-specific siRNA against the S619L mutation or the 2 SNPs (n=50-70 cells tracked). C. Mean speed of cells after 48 hours transfection with 100 nM scramble siRNA (sc) or allele-specific siRNA against the SNP1 (n=50-70 cells tracked). D. Adhesion under basal conditions (n=8 independent assays). E. Adhesion assay after 48 hours transfection with 30 nM scramble siRNA (sc) or allele-specific siRNA against the S619L mutation or the SNP1 (n=5 or 6 independent transfections). F. Adhesion assay after 48 hours transfection with 100 nM scramble siRNA (sc) or allele-specific siRNA against the S619L mutation or SNP1 (n=6 independent transfections). Data information: bars represent mean values and error bars indicate SEM. Statistical analysis are performed using Kruskal-Wallis for migration assay A to C (**** p<0.0001 in all histograms), followed by Dunn's test relative to control cells 1 (*** P<0.001) or control cells 2 (φφP<0.01) in A, or to control cells transfected with scramble siRNA in B and C (**** P<0.0001), or to patient cell lines transfected with scramble siRNA in B and C (††††P<0.0001). Anova tests were performed for adhesion assay in A (**P<0.01), B and C (****P<0.0001) followed by post-tests relative to control cells in A (**P<0.01), to control cells transfected with scramble siRNA (****P<0.0001, ***P<0.001 **P<0.01) and to patient cell lines transfected with scramble siRNA (†† p<0.01, †† p<0.05) in B and C

### EXAMPLE

### Material & Methods

### Cell Cultures and Transfection

Healthy controls and CNM patient-derived fibroblast cell lines were obtained from the MyoLine platform for the immortalization of human cells (Institute of Myology, Paris, France) in accordance with European recommendations and French legislation. Cell lines were cultured at 37 °C (5% CO₂) in Dulbecco's modified Eagle's medium (DMEM, Life Technologies, France) containing 10% fetal calf serum (FCS) supplemented with Penicillin (100 Units/ml) and Streptomycin (100 μg/ml). Patient and control fibroblasts were immortalized using a lentiviral vector containing the sequence encoding the catalytic subunit of human telomerase (hTERT) as previously described⁴⁵. For transfection, cells were grown to 70% confluency and transfected with siRNAs using RNAimax transfection reagent (Life Technologies, France) according to the manufacturer's protocol. The concentration of siRNAs for each experiment was indicated in corresponding figure legends. Allele-specific siRNA and scramble siRNA were purchased from Eurogentec (Belgium) and the sequences are available on request. Cells were used for functional evaluation or harvested for RNA and protein extraction 48 h after transfection.

Total RNA Extraction and cDNA Analysis

Total RNAs were isolated from cells using NucleoSpin RNA (Macherey-Nagel, France) according to the manufacturer's protocol. Cells were passed through a pipetting up-down several times for disruption in the lysis buffer. Total RNAs (500 ng) were submitted to reverse transcription using the Superscript III reverse transcriptase kit (Life Technologies, France) using oligo-dT primers in a final volume of 20 μl. Reverse transcription was performed at 50 °C for 50 minutes, and a final step of 85 °C for 5 minutes was added. To determine the allelic version of each SNP present on the mutated allele, PCR encompassing SNPs and the mutation was performed and cloned using the pGEMT vector system (Promega) and about ten single clones were sequenced (Eurofins, France). The DNM2 expression was quantified by semi-quantitative RT-PCR relative to the HPRT housekeeping gene expression. RT product (1 μl) was submitted to PCR performed at 96 °C for 3 minutes followed by 27 cycles including denaturation at 96 °C for 25 seconds, annealing at 58 °C for 25 seconds and polymerization at 72 °C for 40 seconds and a final step at 72 °C for 5 minutes. The number of 27 PCR cycles has been selected to have the amplification in the exponential range for DNM2 and HPRT. To quantify allele-specificity of the assessed siRNA, assays were developed for SNP1 and SNP2 using restriction enzymes allowing discrimination between the 2 alleles after digestion of the RT-PCR products. PCR was designed to amplify regions of the DNM2 transcript encompassing the SNPs. RT product (1 μl) was submitted to SNP1 or SNP2 PCR performed at 96 °C for 3 minutes followed by 40 cycles including denaturation at 96 °C for 25 seconds, annealing at 58 °C for 25 seconds and polymerization at 72 °C for 40 seconds and a final step at 72 °C for 5 minutes. The number of cycles has been selected to be at the end of the exponential phase of amplification. Ten μl out of the 20 μl PCR products were digested overnight at 37 °C using 7 units of BglI (New England Biolabs, France) for SNP1, and 15 μl out of the 30 μl PCR products were digested overnight at 37 °C using 7 units of Psp5II (New England Biolabs, France) for SNP2. Image acquisition of the PCR products after agarose gel electrophoresis was performed using a Geni2 gel imaging system (Ozyme, France), and the associated signal was quantified using ImageJ Software (NIH; http://rsbweb.nih.gov/ij). All the PCR primers used in this study were from Eurogentec (Belgium) and sequences are available on request.

### Protein Extraction and Western Blot

Cell pellets were homogenized in lysis buffer containing 50 mM of Tris-HCl pH 7.5, 150 mM NaCl, 1% IGEPAL, 0.5% Deoxycholate Sodium and protease inhibitor cocktail 1% (Sigma-Aldrich, France) and kept on rotator for 20 minutes at 4 °C. After cell lysates scraping, samples are lysed by sonication 2 times for 10 seconds at 30% of a maximum power of VCX 130 Vibra-cell ultrasonic processors (Sonics, USA). After centrifugation (12,000 g, 4 °C, 20 minutes), protein concentration in the supernatant was determined with the BCA Protein Assay Kit (Thermo Scientific Pierce, France). Five micrograms of proteins were mixed with a loading buffer (50 mM Tris-HCl, SDS 2%, glycerol 10%, B-mercaptoethanol 1% and bromophenol blue) and denatured at 90 °C for 5 minutes. Protein samples were separated on SDS-PAGE 10% pre-stained gels and transferred onto PVDF membranes (0.45 μm pore size, Life Technologies, France) overnight at 100 mA at 4 °C or nitrocellulose membranes (BioRad Turbo transfer System) by trans blot at 25V, 2.5 mA for 8 minutes. Membranes were blocked for 2 hours at room temperature in PBS containing non-fat dry milk 5% and Tween-20 0.1% and then exposed to rabbit polyclonal anti-Dynamin 2 antibody (ab3457 or ab65556, Abcam, France) or rabbit polyclonal anti-Transferrin receptor antibody (ab84036, Abcam, France) or rabbit polyclonal anti-GAPDH antibody (sc-25778 Santa Cruz, France) in PBS-Tween-20 0.1%, non-fat dry milk 1% overnight at 4 °C. Membranes were rinsed in PBS-Tween-20 0.1% and incubated 2 hours with horseradish peroxidase-conjugated secondary antibody (anti-rabbit from Jackson ImmunoResearch, United Kingdom) in PBS-Tween-20 0.1%. Chemiluminescence was detected using ECL detection Kit (Merck-Millipore, Germany) in a BioRad Chemidoc MP imaging system and signal quantification was performed using ImageJ software.

### Transferrin Uptake Assay and Cell Surface

Transfected cells were cultured in DMEM at 37 °C for 45 minutes. Transferrin-AlexaFluor488 (Life Technologies, France) was added at 20 μg/ml at 37 °C for 15 minutes. Cells were then washed in DMEM pH 2 and PBS and fixed in paraformaldehyde 4% for 15 minutes. Images z-stacks were acquired using an Axio Observer Apotome.2 microscope (Zeiss, Germany) using a 20× Plan Apochromat Zeiss objective. Individual cells were outlined manually on the sum projection of the confocal stacks to measure cell surface (μm2) and the transferrin uptake was calculated using the ImageJ software for each cell according to the formula: total corrected cell fluorescence (CTCF)=integrated density of the cell−(mean fluorescence of background×cell area)

### Adhesion Assay

Cells were harvested by trypsinization 48 hours after transfection or under basal conditions and 35000 cells were seeded on a 12-millimetre diameter glass coverslip in 24-well-plates (3 technical replicates for each transfection). After 1 hour incubation at 37 °C in DMEM-10% FCS supplemented with Penicillin (100 Units/ml) and Streptomycin (100 μg/ml), the non-attached cells were removed by PBS washing and cells attached on coverslips were fixed in paraformaldehyde 4% for 15 minutes. Glass coverslips were mounted on slides with Vectashield medium (Vector Laboratories) containing DAPI to stain nuclei. Images were acquired using an Axio Observer Apotome.2 microscope (Zeiss, Germany) using a 2.5× Plan NeoFluar Zeiss objective and the number of attached cells per μm2 of coverslip were counted using ImageJ software through the counting of nuclei. Three independent experiments were done for each condition.

### Cell Tracking and Analysis

Cells were seeded at low density on 24-wells plastic plates under basal conditions or 48 hours after transfection. Bright-field images were acquired at 10× magnification every 20 or 30 minutes (for transfected cells and basal condition, respectively) for 65 hours using an inverted video-microscope equipped with heat and CO₂ controlled chamber (Nikon Ti2, Oko-Lab) driven by NIS (Nikon). After reconstruction of a 24-hour movie, 50 to 70 cells per condition were randomly chosen and manually tracked by following the position of the nuclei using ImageJ software and MTrackJ plugin to determine mean and max speed⁴⁶. Others motility parameters (mean speed in motion, number and duration of pause) were obtained using Skypad Microsoft Excel add-in that automatically analyzes particles in 2D trajectories⁴⁷. A threshold of 10 μm was set out to consider the cell in movement between two-time frames and a speed threshold of 0.1 μm/minute was set out to consider a relevant displacement.

### Data Analysis and Statistics

Graphics and statistical analyses were performed with GraphPad Prism software versions 6 or 9 (GraphPad Software, LaJolla, California, USA). Values were expressed as means±SEM. The number of samples (n), represents the number of independent biological replicates, as indicated in the figure legends. Number of values analyzed, mean and SEM for all the graph presented in the figures are listed as supplementary table 1.

We used non-parametric statistical tests to analyze our data when the normality could not be assumed (Shapiro-Wilk test) or tested (n too small). In this case, statistical comparisons between groups were performed using unpaired two-tailed Mann-Whitney U-test for the siRNAs screening or using a Kruskal-Wallis test followed when significant by Dunn's for transferrin uptake, cell size and migration assays. Adhesion assay was analyzed using Anova followed by Bonferroni post-test or Welch's ANOVA to correct the inequality of variance followed Dunnett's as post-test. P values indicated for Post hoc tests are adjusted p value. P<0.05 were considered as statistically significant.

### Results

### Identification of Targetable SNPs and Heterozygous Cells

We performed an in-silico analysis devoted to identifying DNM2 SNPs exhibiting the highest frequency of heterozygosity in Human. We looked in Ensembl Genome Browser (https://www.ensembl.org—human genome assembly GRCh38.p13) for synonymous variants in the DNM2 open reading frame or variants in the 5′- and 3′-untranslated transcribed regions (UTR) exhibiting a frequency of the second most common allele between 0.1 and 0.5 in the general population. Using these criteria, we identified two SNPs (data not shown). The first SNP was a T/C variation (rs2229920, thereafter called SNP1) identified as a synonymous variant of the Alanine 713 (c.2139T>C, NM_001005361.3) of the DNM2 protein. Allele frequencies for SNP1 were determined at 0.688 for the allele T and 0.321 for the allele C on 250 390 counts (gnomAD exomes r2.1.1) resulting in a theorical heterozygous (HTZ) frequency of 0.43 (calculated as 2× frequency of T allele x frequency of C allele). In agreement, PCR and Sanger sequencing in a cohort of 52 CNM patients identified 42.3% of SNP1 heterozygosity. The second SNP was an A/T variation (rs12461992, thereafter called SNP2) identified as a 3′-UTR variant located 268 nucleotides after the stop codon (*268A>T, NM_001005361.3). Allele frequencies for SNP2 were determined at 0.824 for the allele A and 0.176 for the allele T resulting in a theorical HTZ frequency of 0.29 on 143 008 counts (gnomAD exomes r3.0) and 23% of SNP2 heterozygosity was found in our cohort of patient. No clinical sign was associated with SNP1 and SNP2.

We next thought to identify cells harbouring the SNPs at HTZ state which were required to screen for allele-specific siRNA against the SNPs. RT-PCR products encompassing the SNPs were amplified from fibroblast cell lines from 2 healthy controls and 2 CNM patients. Sanger sequencing of these RT-PCR products led to identifying one healthy control cell line heterozygous for the 2 SNPs, one CNM cell line (p.R522H DNM2 mutation) harbouring the 2 SNPs at HTZ state, and one CNM cell line (p.S619L DNM2 mutation) harbouring only the SNP1 at HTZ state (data not shown). Through RT-PCR, cloning, and Sanger sequencing of a single allele, we determined that in the R522H cell line, the mutated mRNA also harboured the C version of SNP1 and T version of SNP2 and that in the S619L cell line, the mutated mRNA harboured the C version of SNP1 (data not shown). Altogether, we identified 2 SNPs targetable by AS-RNAi, one healthy control cell line to perform the siRNA screening, and 2 patient-derived cell lines for the functional evaluation of the identified AS-siRNA.

Allele-Specific siRNA Against the C Allele of SNP1

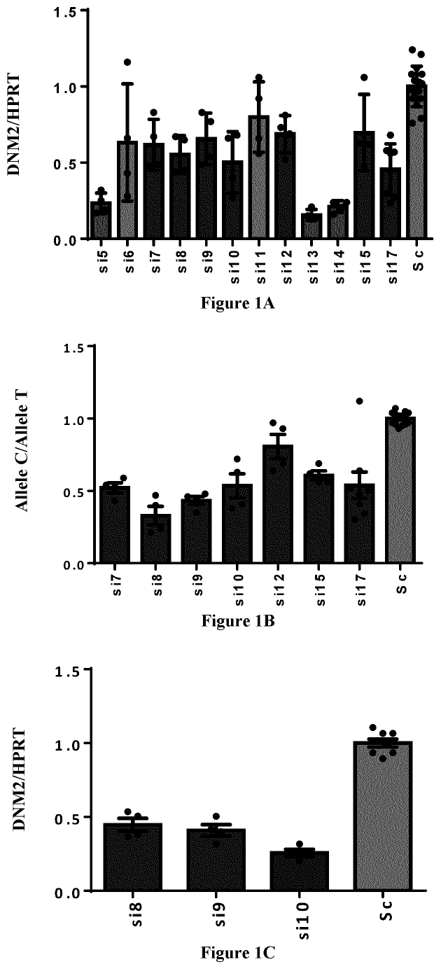

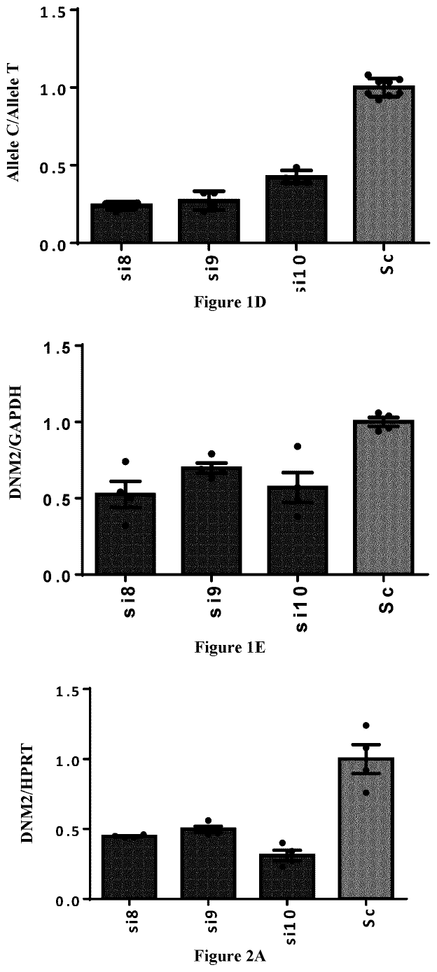

For all the screening for allele-specific siRNA against SNP1 and SNP2 reported in this study, we used one healthy control cell line and looked for allele-specific siRNA leading to around 50% expression of DNM2 mRNA and protein due to specific silencing of the targeted allele. We first thought to identify AS-siRNA against the C version of the SNP1 (FIG. 1). Twelve 19-nucleotide siRNA carrying a single mismatch with the untargeted T version on the positions 5 to 17 (called si5 to si17) were assessed. At low concentration (30 nM), si6 and si11 were not able to significantly reduce DNM2 mRNA expression compared to scramble siRNA-transfected cells (FIG. 1A). In addition, si5, si13, and si14 led to an excessive reduction of DNM2 transcript relative to the expected 50% and 7 siRNAs (si7, si8, si9, si10, si12, si15, and si17) reduced the DNM2 expression in the expected range (FIG. 1A). RT-PCR and BglI restriction enzyme digestion assay was used to discriminate the C (digested) from the T (undigested) alleles of SNP1 (data not shown). The 7 siRNA reduced the C/T ratio compared to scramble siRNA (FIG. 1B) with the lower ratio reached by si8 and si9. Quantification of each allele relative to HPRT mRNA showed that si8, si9, and si10 reduced expression of the allele C without affecting the T (data not shown). At the same concentration, si8, si9, and si10 also reduced expression of the DNM2 protein around the expected 50% compared to scramble siRNA in western blot (data not shown). The maintenance of allele-specificity at higher concentration (100 nM) was then assessed for si8, si9, and si10. The 3 siRNA reduced total DNM2 mRNA content, and close to the expected 50% decrease for si8 and si9 (FIG. 1C). Allele-specificity of si8 and si9 against the targeted C allele was maintained as demonstrated by the C/T ratio reduction (FIG. 1D) and confirmed by quantification of each allele relative to HPRT expression (data not shown). Maintenance of efficacy and allele-specificity of si8 and si9 at higher concentration was also demonstrated on DNM2 protein by western blot showing a significant decrease in DNM2 protein content which did not exceed 50% (FIG. 1E). Altogether, these data validate si8 and si9 as the best allele-specific siRNA against the C version of the SNP1.

Allele-Specific siRNA Against the T Allele of the SNP1

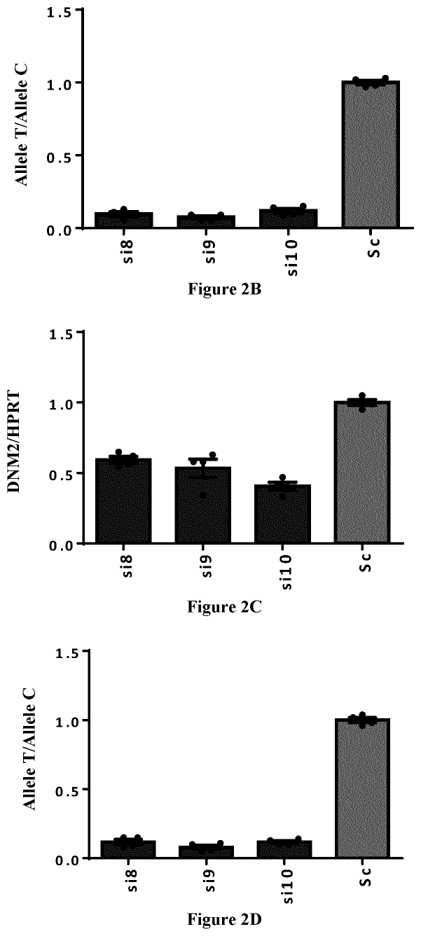

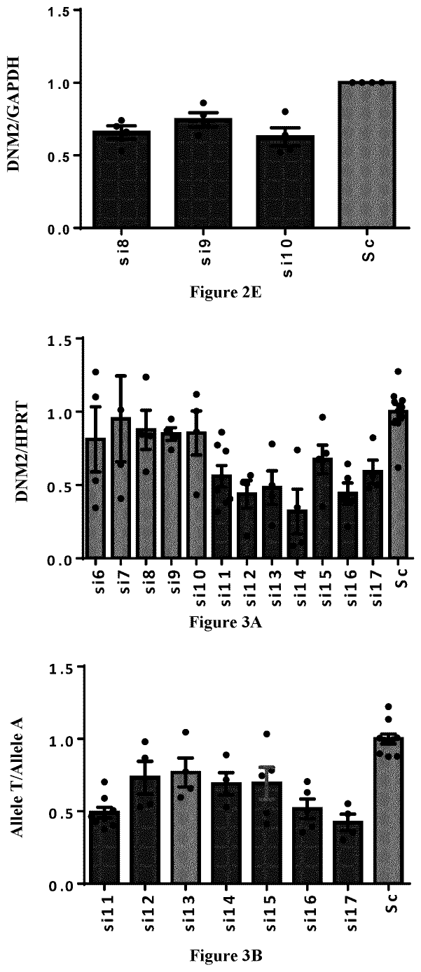

To identify AS-siRNA against the T version of SNP1, we assessed siRNA with mismatches at positions 8, 9, and 10, relative to the untargeted C allele in agreement with our previous screening. At low concentration (30 nM), si8 and si9 significantly reduced the DNM2 expression in the expected range (FIG. 2A). The RT-PCR assay and BglI digestion used to distinguish the targeted T allele from the untargeted C allele showed that the 3 siRNA significantly reduced the T/C ratio compared to scramble siRNA (FIG. 2B). Of note, an increase in the untargeted allele C was observed with si9 (data not shown) suggestive of a genetic regulation promoting the expression of the untargeted allele. At this concentration, the DNM2 protein content was not changed with these 3 siRNA (data not shown). At higher concentration (100 nM), total DNM2 mRNA amount was reduced for the 3 siRNAs remaining close to the 50% threshold for si8 and si9 (FIG. 2C), and allele-specificity of the 3 siRNAs against the targeted T allele was maintained as demonstrated by the T/C ratio reduction (FIG. 2D) and the quantification of each allele relative to HPRT expression (data not shown). A significant increase in the untargeted allele was still observed (data not shown) for the si9 and si10. At this concentration, western blot showed a significant decrease in DNM2 protein content for the si8, si9, and si10 compared to scramble siRNA (FIG. 2E). Altogether, these data validate si8 and si9 as allele-specific siRNAs against the T version of the SNP1.

Allele-Specific siRNA Against the T Allele of the SNP2

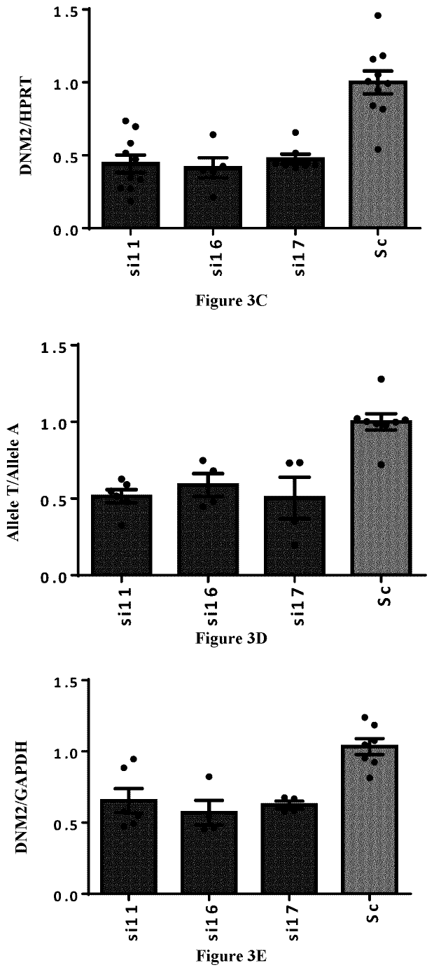

To screen for AS-siRNA against the T version of SNP2, twelve siRNA carrying a mismatch with the untargeted A allele from positions 6 to 17 were assessed (FIG. 3). Transfected at 30 nM for 48h, si11, si12, si13, si14, si15, si16, and si17 reduced DNM2 mRNA expression compared to scramble siRNA-transfected cells, and with the exception of si14 and si15 they all led to a reduction close to 50% (FIG. 3A). RT-PCR and Psp5II restriction enzyme digestion assay was used to discriminate the T digested allele from the A undigested allele (FIG. 3B). Using this assay, we showed that the lower T/A ratio compared to scramble siRNA was reached by si11, si16, and si17 (FIG. 3B). Quantification of each allele relative to HPRT mRNA showed that reduced T/A ratio resulted from a decrease in the expression of the T allele without reducing the A (data not shown). For si17, a significant increase in the A allele was also noticed (data not shown). At this low concentration, only si11 and si16 reduced expression of the DNM2 protein around the expected 50% when compared to scramble siRNA (data not shown). We next assessed the maintenance of allele-specificity for si11, si16, and si17 at higher concentration (100 nM). Semi-quantitative RT-PCR showed a significant reduction of around 50% of total DNM2 mRNA for each siRNA (FIG. 3C). The 3 siRNA reduced the T/A ratio compared to scramble siRNA (FIG. 3D) through a specific impact on the T allele for si11 and si16 (data not shown). At this higher concentration, DNM2 protein content did not exceed 50% (FIG. 3E). Altogether, these data validate si11 and si16 as efficient allele-specific siRNA against the T version of the SNP2.

Allele-Specific siRNA Against the A Allele of SNP2

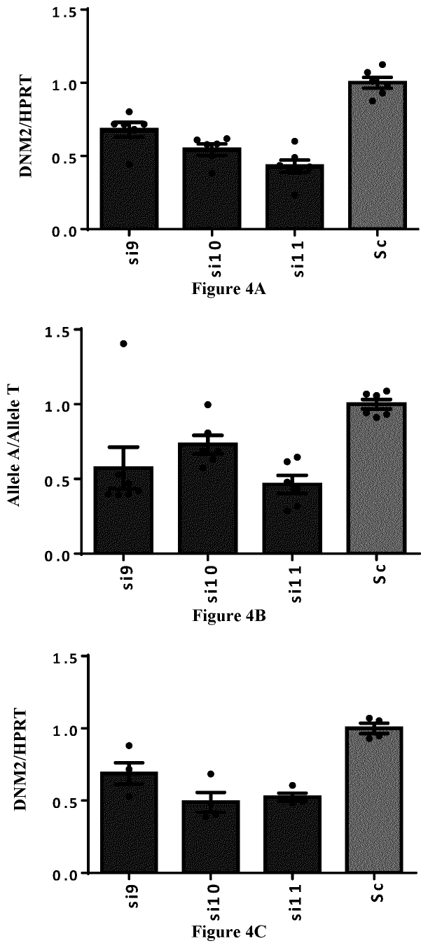

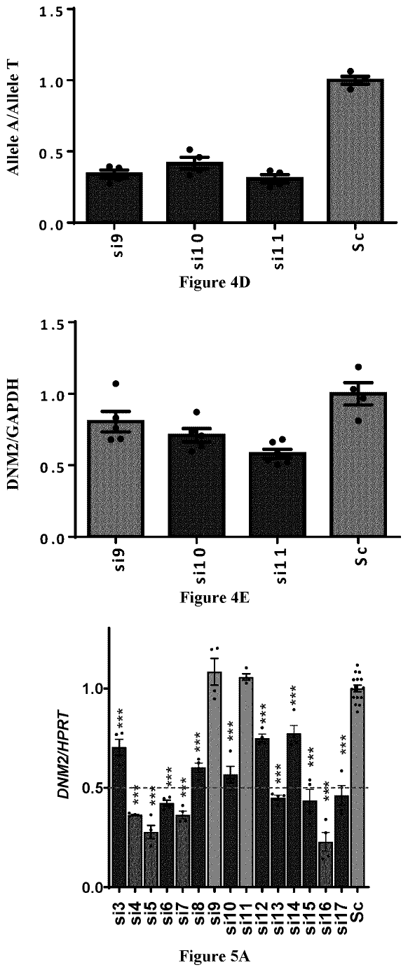

Previous results on the T allele of SNP2 showed a large number of mismatch positions in the siRNAs efficient to develop AS-siRNA. Consequently, we started the screening for AS-siRNA against the A allele by introducing one mismatch in the central region of the siRNA (positions 9, 10, and 11) frequently showed with the highest specificity in previously reported studies (FIG. 4) (23). At low concentration (30 nM), si9, si10, and si11 significantly reduced the DNM2 expression in the expected 50% range (FIG. 4A). The RT-PCR assay and Psp5II digestion used to distinguish the targeted A allele from the untargeted T allele showed that the 3 siRNA significantly reduced the A/T ratio compared to scramble siRNA through the highest impact on the A allele for si9 and si11 (FIG. 4B). At this concentration, the DNM2 protein content was significantly reduced with si10 and si11 (data not shown). At higher concentration (100 nM), a significant reduction of total DNM2 mRNA was observed for the 3 siRNAs (FIG. 4C) and Psp5II digestion of the RT-PCR products showed maintenance of allele-specificity (FIG. 4D). Quantification of each allele relative to HPRT expression showed a reduction of the A allele using si9 and si11 and an upregulation of the untargeted allele T with the 3 siRNA (data not shown). At this concentration, DNM2 protein content, quantified by western blot, was reduced by si10 and si11 (FIG. 4E) but not by si9 probably due to the upregulation of the untargeted T allele (data not shown). Altogether, these data validate si11 as the most efficient allele-specific siRNA against the A version of the SNP2.

Allele-Specific siRNA Against the p.S619L DNM2 Mutation

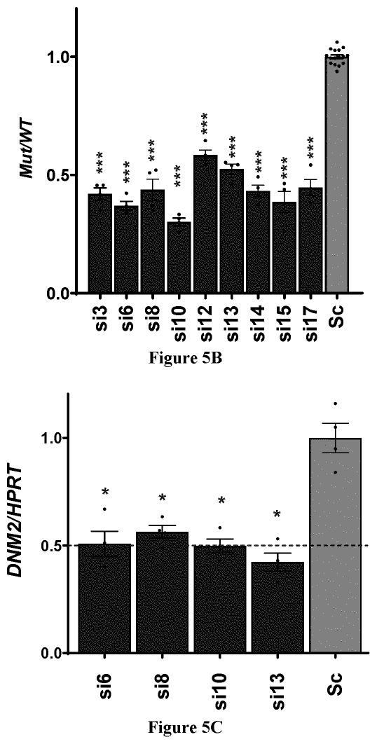

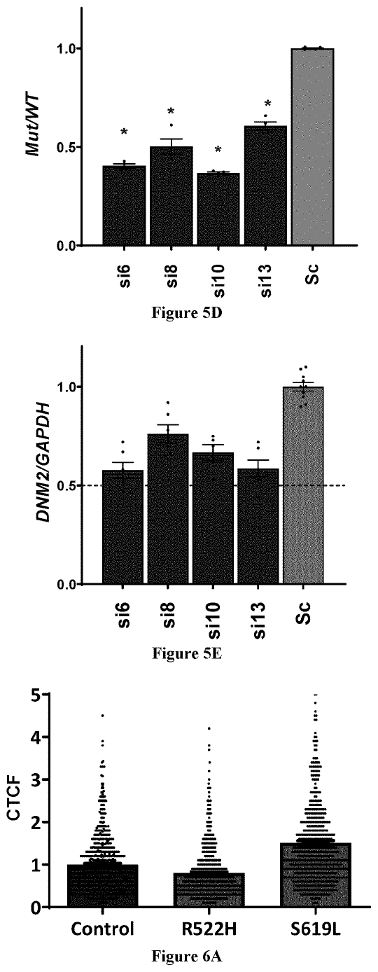

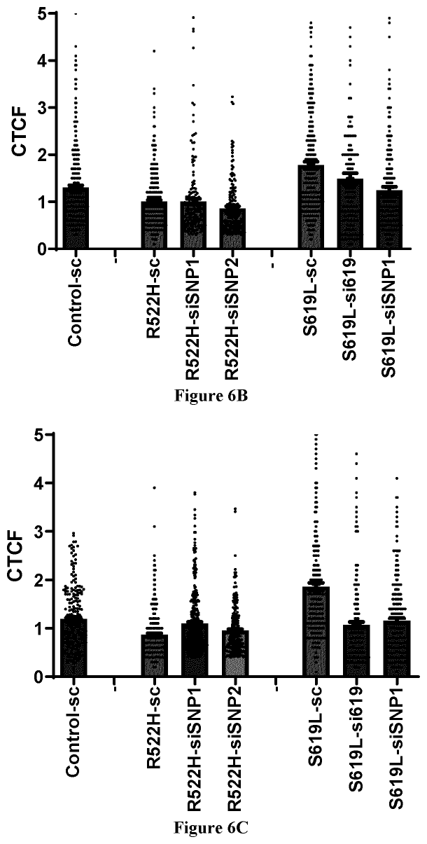

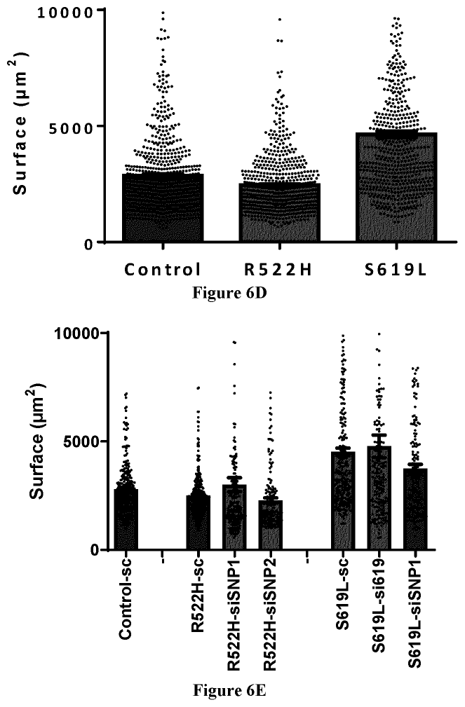

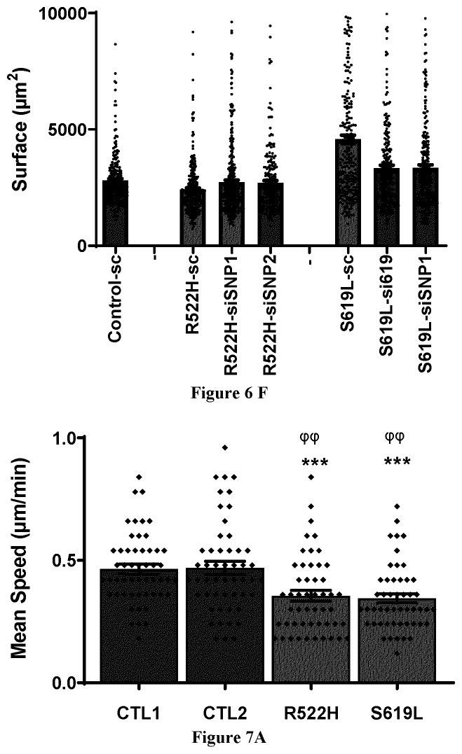

In order to compare the functional benefit of siRNA directed against the SNPs or a mutation, we screened for AS-siRNA against the p.S619L (c.C1856T) (FIG. 5). The S619L patient-derived fibroblasts were used to screen for 15 siRNA (si3 to si17) named depending on the position of the mismatch with the wild-type (WT) sequence of the DNM2 mRNA. At low concentration (30 nM), si9 and si11 were not able to significantly reduce DNM2 mRNA expression compared to scramble siRNA and si4, si5, si7 and si16 led to an excessive reduction of DNM2 transcript relative to the 50% expected (FIG. 5A). Nine siRNAs (si3, si6, si8, si10, si12, si13, si14, si15, and si17) significantly reduced the DNM2 expression, and among them, the si6, si8, si10, si13, si15, si17 reduced mRNA amount in the expected range around 50% (FIG. 5A). Given that the S619L mutation does not introduce or remove a restriction site relative to the WT sequence, we used the presence of the SNP1 at the heterozygous state in this patient cell line to quantify allele-specificity of the 9 siRNAs using BglI digestion of the SNP1 sequence (the C allele of SNP1 and the DNM2 mutation carried by the same mRNA, data not shown). The 9 siRNA significantly reduced the C/T (i.e. the mutant/WT) ratio compared to scramble siRNA (FIG. 5B) with the lower ratio reached by si10. Quantification of each allele relative to HPRT mRNA showed that the 9 siRNAs reduced expression of the mutated allele with some of them inducing increased in the WT allele (Supp FIG. 6A). We assessed the impact on the DNM2 protein content of 4 siRNAs out of the 15 (si6, si8, si10, and si13) and showed a significant reduction which did not exceed 50% for the 4 siRNAs (data not shown). At higher concentration (100 nM), a significant reduction of total DNM2 mRNA was observed for the 4 siRNA (FIG. 5C) and allele-specificity was maintained (FIG. 5D) with the maximum impact on the mutated mRNA reached with the si10. At this concentration, DNM2 protein content, quantified by western blot, was significantly reduced by the 4 siRNAs and the decrease did not exceed 50% (FIG. 5E). Altogether, these data validate 4 allele-specific siRNA (si6, si8, si10 and si13) against the S619L DNM2 mutant allele.

### Rescue of Clathrin-Mediated Endocytosis and Cell Surface by AS-siRNA

DNM2 is well recognized for its role in endocytosis and the defect of clathrin-mediated endocytosis (CME) was previously demonstrated in AD-CNM patient-derived fibroblasts (18,29). We assessed CME through fluorescent transferrin uptake measurement in the healthy control and the 2 DNM2-CNM fibroblast cell lines. Under basal conditions, CME was decreased in fibroblasts harbouring the R522H mutation (R522H-fibroblasts) and increased in fibroblasts harbouring the S619L mutation (S619L-fibroblasts) compared to control fibroblasts (FIG. 6A) and western blot showed similar expression of transferrin receptor between control and mutant cells (data not shown). Among the AS-siRNA identified by our in vitro screening, we used si8 against the C version of the SNP1 (SNP1-si8-C, thereafter called siSNP1), si11 against the T version of the SNP2 (SNP2-si11-T, thereafter called siSNP2), and si10 against the p. S619L mutation (S619L-si10, thereafter called siS619) for the functional evaluation (data not shown). The decrease in transferrin uptake noticed in R522H-fibroblasts was unchanged by siSNP1 and siSNP2 when transfected at 30 nM for 48 hours (FIG. 6B). At 100 nM, the values of transferrin uptake were increased by siSNP1 and siSNP2 close to the control values for siSNP1 after 48 hours (FIG. 6C). For the S619L-fibroblasts, the increase in transferrin was significantly reduced by siS619L and siSNP1 transfected at 30 nM and this is maintained at 100 nM (FIGS. 6B and C).

By measuring cell surface for transferrin uptake quantification, a defect was evidenced with a significantly smaller size for the R522H-fibroblasts and bigger size for the S619L-fibroblasts compared to the control cell line (FIG. 6D). The impact of transfection of the 3 AS-siRNA of interest was then evaluated on the cell surface changes in patient-derived cells (FIGS. 6E and 6F). Among the tested AS-siRNA, siSNP2 improved the cell size of the R522H-fibroblasts when transfected at 100 nM. For the S619L-fibroblasts, siSNP1 improved the cell size from 30 nM and siS619L was able to restore cell size only when transfected at 100 nM. Overall, AS-siRNA directed against non-pathogenic SNPs were found to be as effective as AS-siRNA directly targeting the mutated nucleotide to restore CME and cell surface in patient-derived cells harbouring two distinct DNM2 mutations.

### Rescue of Cell Migration by AS-siRNA

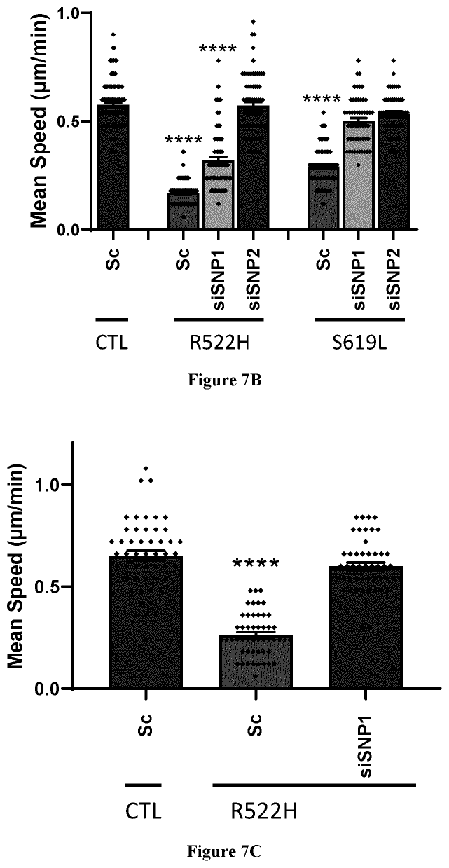

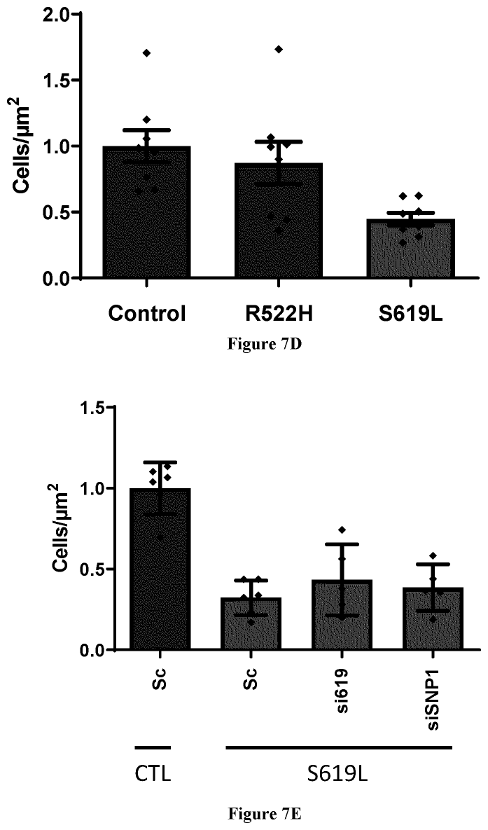

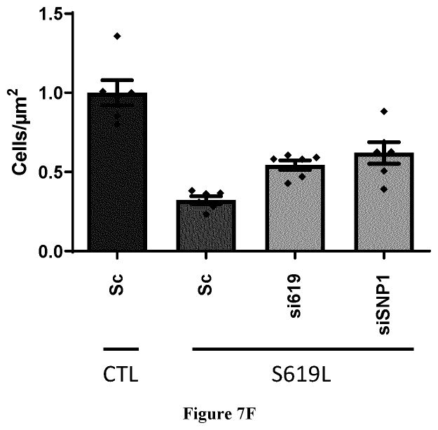

DNM2 dysfunction through its overexpression is known to promote cell migration, invasion and metastasis in cancers 12. Therefore, we looked for migration defects in the 2 fibroblast cell lines carrying the p.R522H and the p. S619L CNM mutations. We have tracked single cells during 24 h and compared the motility behaviour in the 2 patient-derived cell lines and 2 healthy control cell lines. Both controls cells present similar motility behaviour (FIG. 7A) but mutated fibroblasts presented cell motility defects including a reduced mean speed (FIG. 7A), an increased duration of pause and a reduced speed when moving only for the S619L-fibroblasts (data not shown).

To assess the benefit of allele-specific siRNA, we performed the same experiment 48 h after cell transfection with either scramble siRNA or allele-specific siRNA (data not shown). At the concentration of 30 nM, all measured parameters are impaired in mutant fibroblasts transfected with scramble siRNA compared with the control cell line transfected with a scramble. At this concentration, siSNP1 improved all impaired motility parameters in the R522H-fibroblasts (i.e. mean speed, number and duration of pause and speed in motion). The siSNP2 fully rescued all the parameters at the control values (FIG. 7B). For the S619L fibroblasts, siSNP1 and siS619L were effective to rescue or nearly rescuing the motility parameters to the control values (FIG. 7B). A second set of experiments was performed at 100 nM final concentration to evaluate the possibility of a full restoration of motility parameters for siSNP1 in the R522H cells. As shown in FIG. 7C, 100 nM of siSNP1 rescued all the motility parameters. Altogether these results highlight a decrease in cell motility associated with DNM2-CNM mutations which is restored using allele-specific siRNA.

### Rescue of Cell Adhesion by AS-siRNA

The defects of both plasma membrane turnover due to endocytosis impairment and cell migration evidenced in the DNM2-mutated cell lines (FIGS. 6 and 7) may also suggest an impact of the DNM2 mutations on cell adhesion. We next assessed the adhesion capacity of the 2 CNM fibroblast cell lines by quantifying the number of adherent cells per μm2 1 hour after seeding on glass coverslips. A decrease of cell density around 60% was measured in the S619L-fibroblasts compared to control and no change occurred in the R522H-fibroblasts (FIG. 7D). Consequently, we evaluated the impact of AS-siRNA transfected for 48 hours only in S619L-fibroblasts. At 30 nM, siS619L and siSNP1 were unable to revert the adhesion defect (FIG. 7E) whereas a partial rescue was achieved at 100 nM as intermediate values between patient-derived cells and healthy control cells values were measured (FIG. 7F). Overall, these results showed that adhesion defect may be present in CNM patient-derived cells and that selected AS-siRNA against one SNP or the mutation similarly improve this phenotype.

### REFERENCES

Throughout this application, various references describe the state of the art to which this invention pertains. The disclosures of these references are hereby incorporated by reference into the present disclosure.
1. Romero, N. B., and Bitoun, M. (2011). Centronuclear myopathies. Semin Pediatr Neurol 18, 250-6.
2. Hanisch, F., Muller, T., Dietz, A., Bitoun, M., Kress, W., Weis, J., Stoltenburg, G., and Zierz, S. (2011). Phenotype variability and histopathological findings in centronuclear myopathy due to DNM2 mutations. J Neurol 258, 1085-1090.
3 Bitoun, M., Bevilacqua, J. A., Prudhon, B., Maugenre, S., Taratuto, A. L., Monges, S., Lubieniecki, F., Cances, C., Uro-Coste, E., Mayer, M., et al. (2007). Dynamin 2 mutations cause sporadic centronuclear myopathy with neonatal onset. Ann Neurol 62, 666-70.
4. Bitoun, M., Maugenre, S., Jeannet, P. Y., Lacène, E., Ferrer, X., Laforêt, P., Martin, J. J., Laporte, J., Lochmuller, H., Beggs, A. H., et al. (2005). Mutations in dynamin 2 cause dominant Centronuclear Myopathy. Nature Genet 37, 1207-1209.
5. Zuchner, S., Noureddine, M., Kennerson, M., Verhoeven, K., Claeys, K., De Jonghe, P., Merory, J., Oliveira, S. A., Speer, M. C., Stenger, J. E., et al. (2005). Mutations in the pleckstrin homology domain of dynamin 2 cause dominant intermediate Charcot-Marie-Tooth disease. Nat Genet 37, 289-94.

6 Sambuughin, N., Goldfarb, L. G., Sivtseva, T. M., Davydova, T. K., Vladimirtsev, V. A., Osakovskiy, V. L., Danilova, A. P., Nikitina, R. S., Ylakhova, A. N., Diachkovskaya, M. P., et al. (2015). Adult-onset autosomal dominant spastic paraplegia linked to a GTPase-effector domain mutation of dynamin 2. BMC Neurol 15, 223.
7. Raja, S., Shah, S., Tariq, A., Bibi, N., Sughra, K., Yousuf, A., Khawaja, A., Nawaz, M., Mehmood, A., Khan, M., et al. (2019). Caveolin-1 and dynamin-2 overexpression is associated with the progression of bladder cancer. Oncol Lett.
8. Ren, N., Tian, Z., Sun, H., and Lu, X. (2020). Dynamin 2 Is Correlated with Recurrence and Poor Prognosis of Papillary Thyroid Cancer. Med. Sci. Monit. 26, e924590.
9. Chernikova, S. B., Nguyen, R. B., Truong, J. T., Mello, S. S., Stafford, J. H., Hay, M. P., Olson, A., Solow-Cordero, D. E., Wood, D. J., Henry, S., et al. (2018). Dynamin impacts homology-directed repair and breast cancer response to chemotherapy. J Clin Invest 128, 5307-5321.
10. Eppinga, R. D., Krueger, E. W., Weller, S. G., Zhang, L., Cao, H., and McNiven, M. A. (2012). Increased expression of the large GTPase dynamin 2 potentiates metastatic migration and invasion of pancreatic ductal carcinoma. Oncogene 31, 1228-41.
11. Xu, B., Teng, L. H., Silva, S. D., Bijian, K., Al Bashir, S., Jie, S., Dolph, M., Alaoui-Jamali, M. A., and Bismar, T. A. (2014). The significance of dynamin 2 expression for prostate cancer progression, prognostication, and therapeutic targeting. Cancer Med 3, 14-24.
12. Trochet, D., and Bitoun, M. (2021). A review of Dynamin 2 involvement in cancers highlights a promising therapeutic target. J Exp Clin Cancer Res 40, 238.
13. Cowling, B. S., Chevremont, T., Prokic, I., Kretz, C., Ferry, A., Coirault, C., Koutsopoulos, O., Laugel, V., Romero, N. B., and Laporte, J. (2014). Reducing dynamin 2 expression rescues X-linked centronuclear myopathy. J Clin Invest 124, 1350-63.
14. Heymann, J. A., and Hinshaw, J. E. (2009). Dynamins at a glance. J Cell Sci 122, 3427-31.
15. Ferguson, S. M., and De Camilli, P. (2012). Dynamin, a membrane-remodelling GTPase. Nat Rev Mol Cell Biol 13, 75-88.
16. Durieux, A. C., Prudhon, B., Guicheney, P., and Bitoun, M. (2010). Dynamin 2 and Human diseases. J Mol Med 88, 339-350.
17. Bohm, J., Biancalana, V., Dechene, E. T., Bitoun, M., Pierson, C. R., Schaefer, E., Karasoy, H., Dempsey, M. A., Klein, F., Dondaine, N., et al. (2012). Mutation spectrum in the large GTPase dynamin 2, and genotype-phenotype correlation in autosomal dominant centronuclear myopathy. Hum Mutat 33, 949-59.
18. Bitoun, M., Durieux, A. C., Prudhon, B., Bevilacqua, J. A., Herledan, A., Sakanyan, V., Urtizberea, A., Cartier, L., Romero, N. B., and Guicheney, P. (2009). Dynamin 2 mutations associated with human diseases impair clathrin-mediated receptor endocytosis. Hum Mutat 30, 1419-27.
19. Kenniston, J. A., and Lemmon, M. A. (2010). Dynamin GTPase regulation is altered by PH domain mutations found in centronuclear myopathy patients. EMBO J 29, 3054-67.
20. Wang, L., Barylko, B., Byers, C., Ross, J. A., Jameson, D. M., and Albanesi, J. P. (2010). Dynamin 2 mutants linked to centronuclear myopathies form abnormally stable polymers. J Biol Chem 285, 22753-7.
21. Ferguson, S., Raimondi, A., Paradise, S., Shen, H., Mesaki, K., Ferguson, A., Destaing, O., Ko, G., Takasaki, J., Cremona, O., et al. (2009). Coordinated actions of actin and BAR proteins upstream of dynamin at endocytic clathrin-coated pits. Dev Cell 17, 811-22.
22. Trochet, D., Prudhon, B., Beuvin, M., Peccate, C., Lorain, S., Julien, L., Benkhelifa-Ziyyat, S., Rabai, A., Mamchaoui, K., Ferry, A., et al. (2018). Allele-specific silencing therapy for Dynamin 2-related dominant centronuclear myopathy. EMBO Mol Med 10, 239-253.
23. Trochet, D., Prudhon, B., Vassilopoulos, S., and Bitoun, M. (2015). Therapy for Dominant Inherited Diseases by Allele-Specific RNA Interference: Successes and Pitfalls. Curr Gene Ther 15, 503-10.
24. Leachman, S. A., Hickerson, R. P., Schwartz, M. E., Bullough, E. E., Hutcherson, S. L., Boucher, K. M., Hansen, C. D., Eliason, M. J., Srivatsa, G. S., Kornbrust, D. J., et al. (2010). First-in-human mutation-targeted siRNA phase Ib trial of an inherited skin disorder. Mol Ther 18, 442-6.
25. Golan, T., Khvalevsky, E. Z., Hubert, A., Gabai, R. M., Hen, N., Segal, A., Domb, A., Harari, G., David, E. B., Raskin, S., et al. (2015). RNAi therapy targeting KRAS in combination with chemotherapy for locally advanced pancreatic cancer patients. Oncotarget 6, 24560-70.
26. Lombardi, M. S., Jaspers, L., Spronkmans, C., Gellera, C., Taroni, F., Di Maria, E., Donato, S. D., and Kaemmerer, W. F. (2009). A majority of Huntington's disease patients may be treatable by individualized allele-specific RNA interference. Exp Neurol 217, 312-9.
27. Fiszer, A., Olejniczak, M., Switonski, P. M., Wroblewska, J. P., Wisniewska-Kruk, J., Mykowska, A., and Krzyzosiak, W. J. (2012). An evaluation of oligonucleotide-based therapeutic strategies for polyQ diseases. BMC Mol Biol 13, 6.
28. Takahashi, M., Watanabe, S., Murata, M., Furuya, H., Kanazawa, I., Wada, K., and Hohjoh, H. (2010). Tailor-made RNAi knockdown against triplet repeat disease-causing alleles. Proc Natl Acad Sci USA 107, 21731-6.
29. Ali, T., Bednarska, J., Vassilopoulos, S., Tran, M., Diakonov, I. A., Ziyadeh-Isleem, A., Guicheney, P., Gorelik, J., Korchev, Y. E., Reilly, M. M., et al. (2019). Correlative SICM-FCM reveals changes in morphology and kinetics of endocytic pits induced by disease-associated mutations in dynamin. FASEB J 33, 8504-8518.
30. van Bilsen, P. H., Jaspers, L., Lombardi, M. S., Odekerken, J. C., Burright, E. N., and Kaemmerer, W. F. (2008). Identification and allele-specific silencing of the mutant huntingtin allele in Huntington's disease patient-derived fibroblasts. Hum Gene Ther 19, 710-9.
31. Drouet, V., Ruiz, M., Zala, D., Feyeux, M., Auregan, G., Cambon, K., Troquier, L., Carpentier, J., Aubert, S., Merienne, N., et al. (2014). Allele-specific silencing of mutant huntingtin in rodent brain and human stem cells. PLoS One 9, e99341.
32. Scholefield, J., Watson, L., Smith, D., Greenberg, J., and Wood, M. J. (2014). Allele-specific silencing of mutant Ataxin-7 in SCA7 patient-derived fibroblasts. Eur J Hum Genet.
33. Liu, Y. W., Lukiyanchuk, V., and Schmid, S. L. (2011). Common membrane trafficking defects of disease-associated dynamin 2 mutations. Traffic 12, 1620-33.
34. Sidiropoulos, P. N., Miehe, M., Bock, T., Tinelli, E., Oertli, C. I., Kuner, R., Meijer, D., Wollscheid, B., Niemann, A., and Suter, U. (2012). Dynamin 2 mutations in Charcot-Marie-Tooth neuropathy highlight the importance of clathrin-mediated endocytosis in myelination. Brain 135, 1395-411.
35. Rabai, A., Reisser, L., Reina-San-Martin, B., Mamchaoui, K., Cowling, B. S., Nicot, A.-S., and Laporte, J. (2019). Allele-Specific CRISPR/Cas9 Correction of a Heterozygous DNM2 Mutation Rescues Centronuclear Myopathy Cell Phenotypes. Mol Ther Nucleic Acids 16, 246-256.
36. Burton, K. M., Cao, H., Chen, J., Qiang, L., Krueger, E. W., Johnson, K. M., Bamlet, W. R., Zhang, L., McNiven, M. A., and Razidlo, G. L. (2020). Dynamin 2 interacts with a-actinin 4 to drive tumor cell invasion. MBoC 31, 439-451.
37. Yamada, H., Takeda, T., Michiue, H., Abe, T., and Takei, K. (2016). Actin bundling by dynamin 2 and cortactin is implicated in cell migration by stabilizing filopodia in human non-small cell lung carcinoma cells. International Journal of Oncology 49, 877-886.
38. Zhang, Y., Nolan, M., Yamada, H., Watanabe, M., Nasu, Y., Takei, K., and Takeda, T. (2016). Dynamin2 GTPase contributes to invadopodia formation in invasive bladder cancer cells. Biochemical and Biophysical Research Communications 480, 409-414.
39. Razidlo, G. L., Wang, Y., Chen, J., Krueger, E. W., Billadeau, D. D., and McNiven, M. A. (2013). Dynamin 2 potentiates invasive migration of pancreatic tumor cells through stabilization of the Rac1 GEF Vav1. Dev Cell 24, 573-85.
40. Wong, B. S., Shea, D. J., Mistriotis, P., Tuntithavornwat, S., Law, R. A., Bieber, J. M., Zheng, L., and Konstantopoulos, K. (2019). A Direct Podocalyxin-Dynamin-2 Interaction Regulates Cytoskeletal Dynamics to Promote Migration and Metastasis in Pancreatic Cancer Cells. Cancer Res 79, 2878-2891.
41. Yamada, H., Abe, T., Li, S.-A., Tago, S., Huang, P., Watanabe, M., Ikeda, S., Ogo, N., Asai, A., and Takei, K. (2014). N′-[4-(dipropylamino)benzylidene]-2-hydroxybenzohydrazide is a dynamin GTPase inhibitor that suppresses cancer cell migration and invasion by inhibiting actin polymerization. Biochemical and Biophysical Research Communications 443, 511-517.
42. Baldassarre, M., Pompeo, A., Beznoussenko, G., Castaldi, C., Cortellino, S., McNiven, M. A., Luini, A., and Buccione, R. (2003). Dynamin participates in focal extracellular matrix degradation by invasive cells. Mol Biol Cell 14, 1074-84.
43. Rosse, C., Lodillinsky, C., Fuhrmann, L., Nourieh, M., Monteiro, P., Irondelle, M., Lagoutte, E., Vacher, S., Waharte, F., Paul-Gilloteaux, P., et al. (2014). Control of MT1-MMP transport by atypical PKC during breast-cancer progression. Proceedings of the National Academy of Sciences 111, E1872-E1879.
44. F Almeida, C., Bitoun, M., and Vainzof, M. (2021). Satellite cells deficiency and defective regeneration in dynamin 2-related centronuclear myopathy. FASEB J 35, e21346.
45. Auré, K., Mamchaoui, K., Frachon, P., Butler-Browne, G. S., Lombès, A., and Mouly, V. (2007). Impact on oxidative phosphorylation of immortalization with the telomerase gene. Neuromuscul Disord 17, 368-375.
46. Meijering, E., Dzyubachyk, O., and Smal, I. (2012). Methods for cell and particle tracking. Methods Enzymol 504, 183-200.
47. Cadot, B., Gache, V., and Gomes, E. R. (2014). Fast, multi-dimensional and simultaneous kymograph-like particle dynamics (SkyPad) analysis. PLoS One 9, e89073.

1. An allele specific siRNA (AS-siRNA) that silences expression of only one targeted allele of a heterozygous DNM2 gene, wherein the targeted allele comprises a non-pathological polymorphism rs2229920 (C or T) or rs12461992 (A or T) and/or a disease-causing mutation C.1393OT or C.1856C>T.

2. The AS-siRNA according to claim 1, wherein the targeted allele comprises the non-pathological polymorphism is rs2229920 (C or T) or rs12461992 (A or T).

3. The AS-siRNA according to claim 2, wherein the AS-siRNA comprises a sense strand selected from the group consisting of SEQ ID NO: 1, 3, 5, 7, 9, 11, 13, 15, 17, 19, 21, 23, 25, 27, 29, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149 and 151.

4. The AS-siRNA according to claim 3, wherein the AS-siRNA comprises a sense strand selected from the group consisting of SEQ ID NO: 15, 17, 19, 33, 51, 53, 55, 57, 61, 67, 69, 71, 93, 95, 97, 135, 137, 141, 143, 145 and 147.

5. The AS-siRNA according to claim 1 wherein the targeted allele comprises the disease-causing mutation C.1393OT or C.1856OT.

6. The AS-siRNA according to claim 5, wherein the AS-siRNA comprises a sense strand selected from the group consisting of SEQ ID NO: 153, 155, 157, 159, 161, 163, 165, 167, 169, 171, 173, 175, 177, 179, 181, 183, 185, 187, 189, 191, 193, 195, 197, 199, 201, 203, 205, 215, 217, 223, 225 and 227.

7. The AS-siRNA according to claim 6, wherein the AS-siRNA comprises a sense strand selected from the group consisting of SEQ ID NO: 157, 163, 167, 171, 175, 177, 179, 181, and 185.

8. The AS-siRNA according to claim 1, wherein the AS-siRNA contains dinucleotide overhangs comprising two deoxythymidines (dTdT) on a 3′ end of each sense and antisense strand.

9. A vector encoding an AS-siRNA according to claim 1.

10. An in vitro method for silencing expression of a mutated allele of a DNM2 gene without silencing expression of a wild type allele of the DNM2 gene in a target cell, comprising introducing into said target cell an AS-siRNA according to claim 1 or a vector encoding the AS-siRNA.

11. The in vitro method according to claim 10, wherein the mutated allele comprises a non-pathological polymorphism and a disease-causing mutation.

12. (canceled)

13. A method for treating, in subject in need thereof, a disease associated with overexpression of dynamin 2 comprising administering to the subject a therapeutically effective amount of the AS-siRNA of claim 1 or a vector encoding the AS-siRNA.

14. A method of treating a centronuclear myopathy in a subject in need thereof comprising administering to the subject a therapeutically effective amount of the AS-siRNA of claim 1 or a vector encoding the AS-siRNA.

15. A method for treating muscular dystrophy in a subject in need thereof comprising administering to said subject an effective amount of an AS-siRNA according to claim 1 or a vector encoding the AS-siRNA.

16. The method of claim 10, wherein the target cell is a muscle cell.

17. The method of claim 16, wherein the muscle cell is a patient-derived myoblast.

18. The method of claim 13, wherein the disease associated with overexpression of dynamin 2 is an X-linked myotubular myopathy or cancer.

19. The method of claim 16, wherein the cancer is such as prostate cancer or pancreatic cancer.

20. The method of claim 14, wherein the centronuclear myopathy is an autosomal dominant centronuclear myopathy, T-cell acute lymphoblastic leukemia, Charcot-Marie-Tooth disease (CMT) or Hereditary Spastic Paraplegia (HSP).

21. The method of claim 15, wherein the muscular dystrophy is Duchenne muscular dystrophy.


SEQUENCE LISTING (228 sequences)

SEQ ID NO: 1 - RNA, 19 nt, synthetic construct
  source 1..19: mol_type = other RNA
tgaccaggcacagcggcgg

SEQ ID NO: 2 - RNA, 19 nt, synthetic construct
  source 1..19: mol_type = other RNA
ccgccgctgtgcctggtca

SEQ ID NO: 3 - RNA, 19 nt, synthetic construct
  source 1..19: mol_type = other RNA
ctgaccaggcacagcggcg

SEQ ID NO: 4 - RNA, 19 nt, synthetic construct
  source 1..19: mol_type = other RNA
cgccgctgtgcctggtcag

SEQ ID NO: 5 - RNA, 19 nt, synthetic construct
  source 1..19: mol_type = other RNA
gctgaccaggcacagcggc

SEQ ID NO: 6 - RNA, 19 nt, synthetic construct
  source 1..19: mol_type = other RNA
gccgctgtgcctggtcagc

SEQ ID NO: 7 - RNA, 19 nt, synthetic construct
  source 1..19: mol_type = other RNA
ggctgaccaggcacagcgg

SEQ ID NO: 8 - RNA, 19 nt, synthetic construct
  source 1..19: mol_type = other RNA
ccgctgtgcctggtcagcc

SEQ ID NO: 9 - RNA, 19 nt, synthetic construct
  source 1..19: mol_type = other RNA
cggctgaccaggcacagcg

SEQ ID NO: 10 - RNA, 19 nt, synthetic construct
  source 1..19: mol_type = other RNA
cgctgtgcctggtcagccg

SEQ ID NO: 11 - RNA, 19 nt, synthetic construct
  source 1..19: mol_type = other RNA
tcggctgaccaggcacagc

SEQ ID NO: 12 - RNA, 19 nt, synthetic construct
  source 1..19: mol_type = other RNA
gctgtgcctggtcagccga

SEQ ID NO: 13 - RNA, 19 nt, synthetic construct
  source 1..19: mol_type = other RNA
gtcggctgaccaggcacag

SEQ ID NO: 14 - RNA, 19 nt, synthetic construct
  source 1..19: mol_type = other RNA
ctgtgcctggtcagccgac

SEQ ID NO: 15 - RNA, 19 nt, synthetic construct
  source 1..19: mol_type = other RNA
agtcggctgaccaggcaca

SEQ ID NO: 16 - RNA, 19 nt, synthetic construct
  source 1..19: mol_type = other RNA
tgtgcctggtcagccgact

SEQ ID NO: 17 - RNA, 19 nt, synthetic construct
  source 1..19: mol_type = other RNA
gagtcggctgaccaggcac

SEQ ID NO: 18 - RNA, 19 nt, synthetic construct
  source 1..19: mol_type = other RNA
gtgcctggtcagccgactc

SEQ ID NO: 19 - RNA, 19 nt, synthetic construct
  source 1..19: mol_type = other RNA
ggagtcggctgaccaggca

SEQ ID NO: 20 - RNA, 19 nt, synthetic construct
  source 1..19: mol_type = other RNA
tgcctggtcagccgactcc

SEQ ID NO: 21 - RNA, 19 nt, synthetic construct
  source 1..19: mol_type = other RNA
aggagtcggctgaccaggc

SEQ ID NO: 22 - RNA, 19 nt, synthetic construct
  source 1..19: mol_type = other RNA
gcctggtcagccgactcct

SEQ ID NO: 23 - RNA, 19 nt, synthetic construct
  source 1..19: mol_type = other RNA
gaggagtcggctgaccagg

SEQ ID NO: 24 - RNA, 19 nt, synthetic construct
  source 1..19: mol_type = other RNA
cctggtcagccgactcctc

SEQ ID NO: 25 - RNA, 19 nt, synthetic construct
  source 1..19: mol_type = other RNA
ggaggagtcggctgaccag

SEQ ID NO: 26 - RNA, 19 nt, synthetic construct
  source 1..19: mol_type = other RNA
ctggtcagccgactcctcc

SEQ ID NO: 27 - RNA, 19 nt, synthetic construct
  source 1..19: mol_type = other RNA
tggaggagtcggctgacca

SEQ ID NO: 28 - RNA, 19 nt, synthetic construct
  source 1..19: mol_type = other RNA
tggtcagccgactcctcca

SEQ ID NO: 29 - RNA, 19 nt, synthetic construct
  source 1..19: mol_type = other RNA
atggaggagtcggctgacc

SEQ ID NO: 30 - RNA, 19 nt, synthetic construct
  source 1..19: mol_type = other RNA
ggtcagccgactcctccat

SEQ ID NO: 31 - RNA, 19 nt, synthetic construct
  source 1..19: mol_type = other RNA
catggaggagtcggctgac

SEQ ID NO: 32 - RNA, 19 nt, synthetic construct
  source 1..19: mol_type = other RNA
gtcagccgactcctccatg

SEQ ID NO: 33 - RNA, 19 nt, synthetic construct
  source 1..19: mol_type = other RNA
tcatggaggagtcggctga

SEQ ID NO: 34 - RNA, 19 nt, synthetic construct
  source 1..19: mol_type = other RNA
tcagccgactcctccatga

SEQ ID NO: 35 - RNA, 19 nt, synthetic construct
  source 1..19: mol_type = other RNA
ctcatggaggagtcggctg

SEQ ID NO: 36 - RNA, 19 nt, synthetic construct
  source 1..19: mol_type = other RNA
cagccgactcctccatgag

SEQ ID NO: 37 - RNA, 19 nt, synthetic construct
  source 1..19: mol_type = other RNA
cctcatggaggagtcggct

SEQ ID NO: 38 - RNA, 19 nt, synthetic construct
  source 1..19: mol_type = other RNA
agccgactcctccatgagg

SEQ ID NO: 39 - RNA, 19 nt, synthetic construct
  source 1..19: mol_type = other RNA
cgaccaggcacagcggcgg

SEQ ID NO: 40 - RNA, 19 nt, synthetic construct
  source 1..19: mol_type = other RNA
ccgccgctgtgcctggtcg

SEQ ID NO: 41 - RNA, 19 nt, synthetic construct
  source 1..19: mol_type = other RNA
ccgaccaggcacagcggcg

SEQ ID NO: 42 - RNA, 19 nt, synthetic construct
  source 1..19: mol_type = other RNA
cgccgctgtgcctggtcgg

SEQ ID NO: 43 - RNA, 19 nt, synthetic construct
  source 1..19: mol_type = other RNA
gccgaccaggcacagcggc

SEQ ID NO: 44 - RNA, 19 nt, synthetic construct
  source 1..19: mol_type = other RNA
gccgctgtgcctggtcggc

SEQ ID NO: 45 - RNA, 19 nt, synthetic construct
  source 1..19: mol_type = other RNA
ggccgaccaggcacagcgg

SEQ ID NO: 46 - RNA, 19 nt, synthetic construct
  source 1..19: mol_type = other RNA
ccgctgtgcctggtcggcc

SEQ ID NO: 47 - RNA, 19 nt, synthetic construct
  source 1..19: mol_type = other RNA
cggccgaccaggcacagcg

SEQ ID NO: 48 - RNA, 19 nt, synthetic construct
  source 1..19: mol_type = other RNA
cgctgtgcctggtcggccg

SEQ ID NO: 49 - RNA, 19 nt, synthetic construct
  source 1..19: mol_type = other RNA
tcggccgaccaggcacagc

SEQ ID NO: 50 - RNA, 19 nt, synthetic construct
  source 1..19: mol_type = other RNA
gctgtgcctggtcggccga

SEQ ID NO: 51 - RNA, 19 nt, synthetic construct
  source 1..19: mol_type = other RNA
gtcggccgaccaggcacag

SEQ ID NO: 52 - RNA, 19 nt, synthetic construct
  source 1..19: mol_type = other RNA
ctgtgcctggtcggccgac

SEQ ID NO: 53 - RNA, 19 nt, synthetic construct
  source 1..19: mol_type = other RNA
agtcggccgaccaggcaca

SEQ ID NO: 54 - RNA, 19 nt, synthetic construct
  source 1..19: mol_type = other RNA
tgtgcctggtcggccgact

SEQ ID NO: 55 - RNA, 19 nt, synthetic construct
  source 1..19: mol_type = other RNA
gagtcggccgaccaggcac

SEQ ID NO: 56 - RNA, 19 nt, synthetic construct
  source 1..19: mol_type = other RNA
gtgcctggtcggccgactc

SEQ ID NO: 57 - RNA, 19 nt, synthetic construct
  source 1..19: mol_type = other RNA
ggagtcggccgaccaggca

SEQ ID NO: 58 - RNA, 19 nt, synthetic construct
  source 1..19: mol_type = other RNA
tgcctggtcggccgactcc

SEQ ID NO: 59 - RNA, 19 nt, synthetic construct
  source 1..19: mol_type = other RNA
aggagtcggccgaccaggc

SEQ ID NO: 60 - RNA, 19 nt, synthetic construct
  source 1..19: mol_type = other RNA
gcctggtcggccgactcct

SEQ ID NO: 61 - RNA, 19 nt, synthetic construct
  source 1..19: mol_type = other RNA
gaggagtcggccgaccagg

SEQ ID NO: 62 - RNA, 19 nt, synthetic construct
  source 1..19: mol_type = other RNA
ctcctcagccggctggtcc

SEQ ID NO: 63 - RNA, 19 nt, synthetic construct
  source 1..19: mol_type = other RNA
ggaggagtcggccgaccag

SEQ ID NO: 64 - RNA, 19 nt, synthetic construct
  source 1..19: mol_type = other RNA
ctggtcggccgactcctcc

SEQ ID NO: 65 - RNA, 19 nt, synthetic construct
  source 1..19: mol_type = other RNA
tggaggagtcggccgacca

SEQ ID NO: 66 - RNA, 19 nt, synthetic construct
  source 1..19: mol_type = other RNA
tggtcggccgactcctcca

SEQ ID NO: 67 - RNA, 19 nt, synthetic construct
  source 1..19: mol_type = other RNA
atggaggagtcggccgacc

SEQ ID NO: 68 - RNA, 19 nt, synthetic construct
  source 1..19: mol_type = other RNA
ggtcggccgactcctccat

SEQ ID NO: 69 - RNA, 19 nt, synthetic construct
  source 1..19: mol_type = other RNA
catggaggagtcggccgac

SEQ ID NO: 70 - RNA, 19 nt, synthetic construct
  source 1..19: mol_type = other RNA
gtcggccgactcctccatg

SEQ ID NO: 71 - RNA, 19 nt, synthetic construct
  source 1..19: mol_type = other RNA
tcatggaggagtcggccga

SEQ ID NO: 72 - RNA, 19 nt, synthetic construct
  source 1..19: mol_type = other RNA
tcggccgactcctccatga

SEQ ID NO: 73 - RNA, 19 nt, synthetic construct
  source 1..19: mol_type = other RNA
ctcatggaggagtcggccg

SEQ ID NO: 74 - RNA, 19 nt, synthetic construct
  source 1..19: mol_type = other RNA
cggccgactcctccatgag

SEQ ID NO: 75 - RNA, 19 nt, synthetic construct
  source 1..19: mol_type = other RNA
cctcatggaggagtcggcc

SEQ ID NO: 76 - RNA, 19 nt, synthetic construct
  source 1..19: mol_type = other RNA
ggccgactcctccatgagg

SEQ ID NO: 77 - RNA, 19 nt, synthetic construct
  source 1..19: mol_type = other RNA
agaacccttgacaccatcc

SEQ ID NO: 78 - RNA, 19 nt, synthetic construct
  source 1..19: mol_type = other RNA
ggatggtgtcaagggttct

SEQ ID NO: 79 - RNA, 19 nt, synthetic construct
  source 1..19: mol_type = other RNA
cagaacccttgacaccatc

SEQ ID NO: 80 - RNA, 19 nt, synthetic construct
  source 1..19: mol_type = other RNA
gatggtgtcaagggttctg

SEQ ID NO: 81 - RNA, 19 nt, synthetic construct
  source 1..19: mol_type = other RNA
ccagaacccttgacaccat

SEQ ID NO: 82 - RNA, 19 nt, synthetic construct
  source 1..19: mol_type = other RNA
atggtgtcaagggttctgg

SEQ ID NO: 83 - RNA, 19 nt, synthetic construct
  source 1..19: mol_type = other RNA
accagaacccttgacacca

SEQ ID NO: 84 - RNA, 19 nt, synthetic construct
  source 1..19: mol_type = other RNA
tggtgtcaagggttctggt

SEQ ID NO: 85 - RNA, 19 nt, synthetic construct
  source 1..19: mol_type = other RNA
gaccagaacccttgacacc

SEQ ID NO: 86 - RNA, 19 nt, synthetic construct
  source 1..19: mol_type = other RNA
ggtgtcaagggttctggtc

SEQ ID NO: 87 - RNA, 19 nt, synthetic construct
  source 1..19: mol_type = other RNA
ggaccagaacccttgacac

SEQ ID NO: 88 - RNA, 19 nt, synthetic construct
  source 1..19: mol_type = other RNA
gtgtcaagggttctggtcc

SEQ ID NO: 89 - RNA, 19 nt, synthetic construct
  source 1..19: mol_type = other RNA
gggaccagaacccttgaca

SEQ ID NO: 90 - RNA, 19 nt, synthetic construct
  source 1..19: mol_type = other RNA
tgtcaagggttctggtccc

SEQ ID NO: 91 - RNA, 19 nt, synthetic construct
  source 1..19: mol_type = other RNA
ggggaccagaacccttgac

SEQ ID NO: 92 - RNA, 19 nt, synthetic construct
  source 1..19: mol_type = other RNA
gtcaagggttctggtcccc

SEQ ID NO: 93 - RNA, 19 nt, synthetic construct
  source 1..19: mol_type = other RNA
gggggaccagaacccttga

SEQ ID NO: 94 - RNA, 19 nt, synthetic construct
  source 1..19: mol_type = other RNA
tcaagggttctggtccccc

SEQ ID NO: 95 - RNA, 19 nt, synthetic construct
  source 1..19: mol_type = other RNA
ggggggaccagaacccttg

SEQ ID NO: 96 - RNA, 19 nt, synthetic construct
  source 1..19: mol_type = other RNA
caagggttctggtcccccc

SEQ ID NO: 97 - RNA, 19 nt, synthetic construct
  source 1..19: mol_type = other RNA
aggggggaccagaaccctt

SEQ ID NO: 98 - RNA, 19 nt, synthetic construct
  source 1..19: mol_type = other RNA
aagggttctggtcccccct

SEQ ID NO: 99 - RNA, 19 nt, synthetic construct
  source 1..19: mol_type = other RNA
gaggggggaccagaaccct

SEQ ID NO: 100 - RNA, 19 nt, synthetic construct
  source 1..19: mol_type = other RNA
agggttctggtcccccctc

SEQ ID NO: 101 - RNA, 19 nt, synthetic construct
  source 1..19: mol_type = other RNA
agaggggggaccagaaccc

SEQ ID NO: 102 - RNA, 19 nt, synthetic construct
  source 1..19: mol_type = other RNA
gggttctggtcccccctct

SEQ ID NO: 103 - RNA, 19 nt, synthetic construct
  source 1..19: mol_type = other RNA
cagaggggggaccagaacc

SEQ ID NO: 104 - RNA, 19 nt, synthetic construct
  source 1..19: mol_type = other RNA
ggttctggtcccccctctg

SEQ ID NO: 105 - RNA, 19 nt, synthetic construct
  source 1..19: mol_type = other RNA
gcagaggggggaccagaac

SEQ ID NO: 106 - RNA, 19 nt, synthetic construct
  source 1..19: mol_type = other RNA
gttctggtcccccctctgc

SEQ ID NO: 107 - RNA, 19 nt, synthetic construct
  source 1..19: mol_type = other RNA
ggcagaggggggaccagaa

SEQ ID NO: 108 - RNA, 19 nt, synthetic construct
  source 1..19: mol_type = other RNA
ttctggtcccccctctgcc

SEQ ID NO: 109 - RNA, 19 nt, synthetic construct
  source 1..19: mol_type = other RNA
tggcagaggggggaccaga

SEQ ID NO: 110 - RNA, 19 nt, synthetic construct
  source 1..19: mol_type = other RNA
tctggtcccccctctgcca

SEQ ID NO: 111 - RNA, 19 nt, synthetic construct
  source 1..19: mol_type = other RNA
gtggcagaggggggaccag

SEQ ID NO: 112 - RNA, 19 nt, synthetic construct
  source 1..19: mol_type = other RNA
ctggtcccccctctgccac

SEQ ID NO: 113 - RNA, 19 nt, synthetic construct
  source 1..19: mol_type = other RNA
ggtggcagaggggggacca

SEQ ID NO: 114 - RNA, 19 nt, synthetic construct
  source 1..19: mol_type = other RNA
tggtcccccctctgccacc

SEQ ID NO: 115 - RNA, 19 nt, synthetic construct
  source 1..19: mol_type = other RNA
tgaacccttgacaccatcc

SEQ ID NO: 116 - RNA, 19 nt, synthetic construct
  source 1..19: mol_type = other RNA
ggatggtgtcaagggttca

SEQ ID NO: 117 - RNA, 19 nt, synthetic construct
  source 1..19: mol_type = other RNA
ctgaacccttgacaccatc

SEQ ID NO: 118 - RNA, 19 nt, synthetic construct
  source 1..19: mol_type = other RNA
gatggtgtcaagggttcag

SEQ ID NO: 119 - RNA, 19 nt, synthetic construct
  source 1..19: mol_type = other RNA
cctgaacccttgacaccat

SEQ ID NO: 120 - RNA, 19 nt, synthetic construct
  source 1..19: mol_type = other RNA
atggtgtcaagggttcagg

SEQ ID NO: 121 - RNA, 19 nt, synthetic construct
  source 1..19: mol_type = other RNA
acctgaacccttgacacca

SEQ ID NO: 122 - RNA, 19 nt, synthetic construct
  source 1..19: mol_type = other RNA
tggtgtcaagggttcaggt

SEQ ID NO: 123 - RNA, 19 nt, synthetic construct
  source 1..19: mol_type = other RNA
gacctgaacccttgacacc

SEQ ID NO: 124 - RNA, 19 nt, synthetic construct
  source 1..19: mol_type = other RNA
ggtgtcaagggttcaggtc

SEQ ID NO: 125 - RNA, 19 nt, synthetic construct
  source 1..19: mol_type = other RNA
ggacctgaacccttgacac

SEQ ID NO: 126 - RNA, 19 nt, synthetic construct
  source 1..19: mol_type = other RNA
gtgtcaagggttcaggtcc

SEQ ID NO: 127 - RNA, 19 nt, synthetic construct
  source 1..19: mol_type = other RNA
gggacctgaacccttgaca

SEQ ID NO: 128 - RNA, 19 nt, synthetic construct
  source 1..19: mol_type = other RNA
tgtcaagggttcaggtccc

SEQ ID NO: 129 - RNA, 19 nt, synthetic construct
  source 1..19: mol_type = other RNA
ggggacctgaacccttgac

SEQ ID NO: 130 - RNA, 19 nt, synthetic construct
  source 1..19: mol_type = other RNA
gtcaagggttcaggtcccc

SEQ ID NO: 131 - RNA, 19 nt, synthetic construct
  source 1..19: mol_type = other RNA
gggggacctgaacccttga

SEQ ID NO: 132 - RNA, 19 nt, synthetic construct
  source 1..19: mol_type = other RNA
tcaagggttcaggtccccc

SEQ ID NO: 133 - RNA, 19 nt, synthetic construct
  source 1..19: mol_type = other RNA
ggggggacctgaacccttg

SEQ ID NO: 134 - RNA, 19 nt, synthetic construct
  source 1..19: mol_type = other RNA
caagggttcaggtcccccc

SEQ ID NO: 135 - RNA, 19 nt, synthetic construct
  source 1..19: mol_type = other RNA
aggggggacctgaaccctt

SEQ ID NO: 136 - RNA, 19 nt, synthetic construct
  source 1..19: mol_type = other RNA
aagggttcaggtcccccct

SEQ ID NO: 137 - RNA, 19 nt, synthetic construct
  source 1..19: mol_type = other RNA
gaggggggacctgaaccct

SEQ ID NO: 138 - RNA, 19 nt, synthetic construct
  source 1..19: mol_type = other RNA
agggttcaggtcccccctc

SEQ ID NO: 139 - RNA, 19 nt, synthetic construct
  source 1..19: mol_type = other RNA
agaggggggacctgaaccc

SEQ ID NO: 140 - RNA, 19 nt, synthetic construct
  source 1..19: mol_type = other RNA
gggttcaggtcccccctct

SEQ ID NO: 141 - RNA, 19 nt, synthetic construct
  source 1..19: mol_type = other RNA
cagaggggggacctgaacc

SEQ ID NO: 142 - RNA, 19 nt, synthetic construct
  source 1..19: mol_type = other RNA
ggttcaggtcccccctctg

SEQ ID NO: 143 - RNA, 19 nt, synthetic construct
  source 1..19: mol_type = other RNA
gcagaggggggacctgaac

SEQ ID NO: 144 - RNA, 19 nt, synthetic construct
  source 1..19: mol_type = other RNA
gttcaggtcccccctctgc

SEQ ID NO: 145 - RNA, 19 nt, synthetic construct
  source 1..19: mol_type = other RNA
ggcagaggggggacctgaa

SEQ ID NO: 146 - RNA, 19 nt, synthetic construct
  source 1..19: mol_type = other RNA
ttcaggtcccccctctgcc

SEQ ID NO: 147 - RNA, 19 nt, synthetic construct
  source 1..19: mol_type = other RNA
tggcagaggggggacctga

SEQ ID NO: 148 - RNA, 19 nt, synthetic construct
  source 1..19: mol_type = other RNA
tcaggtcccccctctgcca

SEQ ID NO: 149 - RNA, 19 nt, synthetic construct
  source 1..19: mol_type = other RNA
gtggcagaggggggacctg

SEQ ID NO: 150 - RNA, 19 nt, synthetic construct
  source 1..19: mol_type = other RNA
caggtcccccctctgccac

SEQ ID NO: 151 - RNA, 19 nt, synthetic construct
  source 1..19: mol_type = other RNA
ggtggcagaggggggacct

SEQ ID NO: 152 - RNA, 19 nt, synthetic construct
  source 1..19: mol_type = other RNA
aggtcccccctctgccacc

SEQ ID NO: 153 - RNA, 19 nt, synthetic construct
  source 1..19: mol_type = other RNA
tgttcctccgagctggcgt

SEQ ID NO: 154 - RNA, 19 nt, synthetic construct
  source 1..19: mol_type = other RNA
acgccagctcggaggaaca

SEQ ID NO: 155 - RNA, 19 nt, synthetic construct
  source 1..19: mol_type = other RNA
ttgttcctccgagctggcg

SEQ ID NO: 156 - RNA, 19 nt, synthetic construct
  source 1..19: mol_type = other RNA
cgccagctcggaggaacaa

SEQ ID NO: 157 - RNA, 19 nt, synthetic construct
  source 1..19: mol_type = other RNA
cttgttcctccgagctggc

SEQ ID NO: 158 - RNA, 19 nt, synthetic construct
  source 1..19: mol_type = other RNA
gccagctcggaggaacaag

SEQ ID NO: 159 - RNA, 19 nt, synthetic construct
  source 1..19: mol_type = other RNA
ccttgttcctccgagctgg

SEQ ID NO: 160 - RNA, 19 nt, synthetic construct
  source 1..19: mol_type = other RNA
ccagctcggaggaacaagg

SEQ ID NO: 161 - RNA, 19 nt, synthetic construct
  source 1..19: mol_type = other RNA
gccttgttcctccgagctg

SEQ ID NO: 162 - RNA, 19 nt, synthetic construct
  source 1..19: mol_type = other RNA
cagctcggaggaacaaggc

SEQ ID NO: 163 - RNA, 19 nt, synthetic construct
  source 1..19: mol_type = other RNA
ggccttgttcctccgagct

SEQ ID NO: 164 - RNA, 19 nt, synthetic construct
  source 1..19: mol_type = other RNA
agctcggaggaacaaggcc

SEQ ID NO: 165 - RNA, 19 nt, synthetic construct
  source 1..19: mol_type = other RNA
aggccttgttcctccgagc

SEQ ID NO: 166 - RNA, 19 nt, synthetic construct
  source 1..19: mol_type = other RNA
gctcggaggaacaaggcct

SEQ ID NO: 167 - RNA, 19 nt, synthetic construct
  source 1..19: mol_type = other RNA
aaggccttgttcctccgag

SEQ ID NO: 168 - RNA, 19 nt, synthetic construct
  source 1..19: mol_type = other RNA
ctcggaggaacaaggcctt

SEQ ID NO: 169 - RNA, 19 nt, synthetic construct
  source 1..19: mol_type = other RNA
gaaggccttgttcctccga

SEQ ID NO: 170 - RNA, 19 nt, synthetic construct
  source 1..19: mol_type = other RNA
tcggaggaacaaggccttc

SEQ ID NO: 171 - RNA, 19 nt, synthetic construct
  source 1..19: mol_type = other RNA
ggaaggccttgttcctccg

SEQ ID NO: 172 - RNA, 19 nt, synthetic construct
  source 1..19: mol_type = other RNA
cggaggaacaaggccttcc

SEQ ID NO: 173 - RNA, 19 nt, synthetic construct
  source 1..19: mol_type = other RNA
tggaaggccttgttcctcc

SEQ ID NO: 174 - RNA, 19 nt, synthetic construct
  source 1..19: mol_type = other RNA
ggaggaacaaggccttcca

SEQ ID NO: 175 - RNA, 19 nt, synthetic construct
  source 1..19: mol_type = other RNA
ctggaaggccttgttcctc

SEQ ID NO: 176 - RNA, 19 nt, synthetic construct
  source 1..19: mol_type = other RNA
gaggaacaaggccttccag

SEQ ID NO: 177 - RNA, 19 nt, synthetic construct
  source 1..19: mol_type = other RNA
gctggaaggccttgttcct

SEQ ID NO: 178 - RNA, 19 nt, synthetic construct
  source 1..19: mol_type = other RNA
aggaacaaggccttccagc

SEQ ID NO: 179 - RNA, 19 nt, synthetic construct
  source 1..19: mol_type = other RNA
agctggaaggccttgttcc

SEQ ID NO: 180 - RNA, 19 nt, synthetic construct
  source 1..19: mol_type = other RNA
ggaacaaggccttccagct

SEQ ID NO: 181 - RNA, 19 nt, synthetic construct
  source 1..19: mol_type = other RNA
cagctggaaggccttgttc

SEQ ID NO: 182 - RNA, 19 nt, synthetic construct
  source 1..19: mol_type = other RNA
gaacaaggccttccagctg

SEQ ID NO: 183 - RNA, 19 nt, synthetic construct
  source 1..19: mol_type = other RNA
acagctggaaggccttgtt

SEQ ID NO: 184 - RNA, 19 nt, synthetic construct
  source 1..19: mol_type = other RNA
aacaaggccttccagctgt

SEQ ID NO: 185 - RNA, 19 nt, synthetic construct
  source 1..19: mol_type = other RNA
gacagctggaaggccttgt

SEQ ID NO: 186 - RNA, 19 nt, synthetic construct
  source 1..19: mol_type = other RNA
acaaggccttccagctgtc

SEQ ID NO: 187 - RNA, 19 nt, synthetic construct
  source 1..19: mol_type = other RNA
ggacagctggaaggccttg

SEQ ID NO: 188 - RNA, 19 nt, synthetic construct
  source 1..19: mol_type = other RNA
caaggccttccagctgtcc

SEQ ID NO: 189 - RNA, 19 nt, synthetic construct
  source 1..19: mol_type = other RNA
tggacagctggaaggcctt

SEQ ID NO: 190 - RNA, 19 nt, synthetic construct
  source 1..19: mol_type = other RNA
aaggccttccagctgtcca

SEQ ID NO: 191 - RNA, 19 nt, synthetic construct
  source 1..19: mol_type = other RNA
tgggaacgggaggggagaa

SEQ ID NO: 192 - RNA, 19 nt, synthetic construct
  source 1..19: mol_type = other RNA
ttctcccctcccgttccca

SEQ ID NO: 193 - RNA, 19 nt, synthetic construct
  source 1..19: mol_type = other RNA
ctgggaacgggaggggaga

SEQ ID NO: 194 - RNA, 19 nt, synthetic construct
  source 1..19: mol_type = other RNA
tctcccctcccgttcccag

SEQ ID NO: 195 - RNA, 19 nt, synthetic construct
  source 1..19: mol_type = other RNA
tctgggaacgggaggggag

SEQ ID NO: 196 - RNA, 19 nt, synthetic construct
  source 1..19: mol_type = other RNA
ctcccctcccgttcccaga

SEQ ID NO: 197 - RNA, 19 nt, synthetic construct
  source 1..19: mol_type = other RNA
atctgggaacgggagggga

SEQ ID NO: 198 - RNA, 19 nt, synthetic construct
  source 1..19: mol_type = other RNA
tcccctcccgttcccagat

SEQ ID NO: 199 - RNA, 19 nt, synthetic construct
  source 1..19: mol_type = other RNA
catctgggaacgggagggg

SEQ ID NO: 200 - RNA, 19 nt, synthetic construct
  source 1..19: mol_type = other RNA
cccctcccgttcccagatg

SEQ ID NO: 201 - RNA, 19 nt, synthetic construct
  source 1..19: mol_type = other RNA
acatctgggaacgggaggg

SEQ ID NO: 202 - RNA, 19 nt, synthetic construct
  source 1..19: mol_type = other RNA
ccctcccgttcccagatgt

SEQ ID NO: 203 - RNA, 19 nt, synthetic construct
  source 1..19: mol_type = other RNA
tacatctgggaacgggagg

SEQ ID NO: 204 - RNA, 19 nt, synthetic construct
  source 1..19: mol_type = other RNA
cctcccgttcccagatgta

SEQ ID NO: 205 - RNA, 19 nt, synthetic construct
  source 1..19: mol_type = other RNA
ttacatctgggaacgggag

SEQ ID NO: 206 - RNA, 19 nt, synthetic construct
  source 1..19: mol_type = other RNA
ctcccgttcccagatgtaa

SEQ ID NO: 207 - RNA, 19 nt, synthetic construct
  source 1..19: mol_type = other RNA
cttacatctgggaacggga

SEQ ID NO: 208 - RNA, 19 nt, synthetic construct
  source 1..19: mol_type = other RNA
tcccgttcccagatgtaag

SEQ ID NO: 209 - RNA, 19 nt, synthetic construct
  source 1..19: mol_type = other RNA
acttacatctgggaacggg

SEQ ID NO: 210 - RNA, 19 nt, synthetic construct
  source 1..19: mol_type = other RNA
cccgttcccagatgtaagt

SEQ ID NO: 211 - RNA, 19 nt, synthetic construct
  source 1..19: mol_type = other RNA
cacttacatctgggaacgg

SEQ ID NO: 212 - RNA, 19 nt, synthetic construct
  source 1..19: mol_type = other RNA
ccgttcccagatgtaagtg

SEQ ID NO: 213 - RNA, 19 nt, synthetic construct
  source 1..19: mol_type = other RNA
ccacttacatctgggaacg

SEQ ID NO: 214 - RNA, 19 nt, synthetic construct
  source 1..19: mol_type = other RNA
cgttcccagatgtaagtgg

SEQ ID NO: 215 - RNA, 19 nt, synthetic construct
  source 1..19: mol_type = other RNA
accacttacatctgggaac

SEQ ID NO: 216 - RNA, 19 nt, synthetic construct
  source 1..19: mol_type = other RNA
gttcccagatgtaagtggt

SEQ ID NO: 217 - RNA, 19 nt, synthetic construct
  source 1..19: mol_type = other RNA
caccacttacatctgggaa

SEQ ID NO: 218 - RNA, 19 nt, synthetic construct
  source 1..19: mol_type = other RNA
ttcccagatgtaagtggtg

SEQ ID NO: 219 - RNA, 19 nt, synthetic construct
  source 1..19: mol_type = other RNA
tcaccacttacatctggga

SEQ ID NO: 220 - RNA, 19 nt, synthetic construct
  source 1..19: mol_type = other RNA
tcccagatgtaagtggtga

SEQ ID NO: 221 - RNA, 19 nt, synthetic construct
  source 1..19: mol_type = other RNA
gtcaccacttacatctggg

SEQ ID NO: 222 - RNA, 19 nt, synthetic construct
  source 1..19: mol_type = other RNA
cccagatgtaagtggtgac

SEQ ID NO: 223 - RNA, 19 nt, synthetic construct
  source 1..19: mol_type = other RNA
cgtcaccacttacatctgg

SEQ ID NO: 224 - RNA, 19 nt, synthetic construct
  source 1..19: mol_type = other RNA
ccagatgtaagtggtgacg

SEQ ID NO: 225 - RNA, 19 nt, synthetic construct
  source 1..19: mol_type = other RNA
tcgtcaccacttacatctg

SEQ ID NO: 226 - RNA, 19 nt, synthetic construct
  source 1..19: mol_type = other RNA
cagatgtaagtggtgacga

SEQ ID NO: 227 - RNA, 19 nt, synthetic construct
  source 1..19: mol_type = other RNA
atcgtcaccacttacatct

SEQ ID NO: 228 - RNA, 19 nt, synthetic construct
  source 1..19: mol_type = other RNA
agatgtaagtggtgacgat